(19) United States
(12) Patent Application Publication
(10) Pub. No.: US 2024/0010706 A1
(43) Pub. Date: Jan. 11, 2024

(54) IMMUNOMODULATORY COMPOSTIONS AND USE THEREOF

(71) Applicant: BIOND BIOLOGICS LTD., Misgav Industrial Park (IL)

(72) Inventors: Ilana MANDEL, Manof (IL); Motti HAKIM, Kibutz Gazit (IL); Zohar KEREN, Gesher Haziv (IL); Yair SAPIR, Manof (IL); Avidor SHULMAN, Rakefet (IL); Tehila BEN-MOSHE, Tel Aviv (IL)

(21) Appl. No.: 17/915,761

(22) PCT Filed: Mar. 31, 2021
(86) PCT No.: PCT/IL2021/050365

§ 371 Date: Sep. 29, 2022

Related U.S. Application Data
(60) Provisional application No. 63/002,467, filed on Mar. 31, 2020.

(51) Int. Cl. C07K 14/75; A61P 37/00

(52) U.S. Cl. CPC C07K 14/75; A61P 37/00; C07K 2319/21; C07K 2319/31; C07K 2319/30

(57) ABSTRACT

Chimeric molecules comprising a fibrinogen-related domain (FRED) from human fibrinogen-like protein 2 (FGL2) are provided. Immunomodulatory pharmaceutical compositions comprising the chimeric molecules, as well as methods for reducing inflammation and treating autoimmune disease in a subject by administering the chimeric molecules or immunomodulatory pharmaceutical compositions are also provided.

### CROSS REFERENCE TO RELATED APPLICATIONS

This application claims the benefit of priority of U.S. Provisional Patent Application No. 63/002,467 filed on Mar. 31, 2020, the contents of which is incorporated herein by reference in its entirety.

### FIELD OF INVENTION

The present invention is in the field of immune regulation.

### BACKGROUND OF THE INVENTION

Fibrinogen-like protein 2 (FGL2), also known as fibroleukin or prothrombinase, is a member of the fibrinogen superfamily (fibrinogen-related domain, FRED) due to its homology with fibrinogen β and γ chains. FGL2 has two structurally different forms: the membrane bound FGL2 (mFGL2) and the soluble FGL2 (sFGL2). mFGL2, a 70 kDa type II transmembrane glycoprotein expressed on the surface of macrophages or endothelial cells, exerts a procoagulant activity in immune-associated coagulation. sFGL2 has a 50 kDa weight and is highly expressed by CD4⁺CD25⁺ regulatory T cells (Tregs) and other Treg populations and may be a common effector molecule of many classes of Tregs.

Fcγ receptor (FcγR) IIB and FcγRIII have been identified as the mediators of sFGL2 function. FcγRIII3 has an immunoreceptor tyrosine-based inhibition motif (ITIM) in its intracytoplasmic domain and is the only FcγR that has an inhibitory function. FcγRIII contains an immunoreceptor tyrosine-based inhibition motif (ITAM) that mediates the activating signaling. After binding to FcγRs, sFGL2 has distinct biological effects on diverse cell types, which may be due to different expression ratios of FcγRIII3 to FcγRIII on cellular surfaces, or different affinities of sFGL2 to those two FcγRs.

It has been recognized that sFGL2 acts as an important effector molecule of CD4⁺CD25⁺ Tregs in their development and function in murine models. It has been shown that CD4⁺CD25⁺ Tregs are more abundant in fgl2−/− mice, but their ability to suppress effector CD4⁺ T cells proliferation is significantly impaired. In contrast to the blockade of IL-10, TGF-β or CTLA-4 which was ineffective or weak in CD4⁺CD25⁺ Treg activity inhibition, administration of a sFGL2 neutralizing antibody abolished the suppressive activity of murine CD4⁺CD25⁺ Tregs in vitro in a dose dependent manner [Shalev, I. et al., J Immunol., 2008, 180:249-260]. More recently, Joller et al. revealed that murine sFGL2 was indispensable for the ability of the novel identified TIGIT⁺CD4⁺CD25⁺ Treg cells subset to suppress Th1 and Th17 cell response [Joller, N. et al., Immunity, 2014, 40:569-581]. Taken together, sFGL2-mediated immunoregulation might be crucial for the maintenance of Th cell homeostasis.

However, most of the studies related to sFGL2's role as an immunoregulator were done in murine models, while there are many studies that highlight the differences between humans and mice regarding T cells regulation. Thus, the role of sFGL2 as an immunoregulator in humans is still not clearly verified.

The fgl2 gene, localized to the proximal region of chromosome 7q11.23 in humans and 5 in mice, is composed of two exons that are separated by one intron. The longest open reading frame (ORF) of FGL2 encodes a protein of 439 amino acids in humans and 432 amino acids in mice. Analysis of the FGL2 protein predicted an N terminal coiled-coil domain and a C terminal globular domain (FRED). Eleven of the 12 cysteines found in mouse FGL2 are present in the human and pig FGL2 analyzed, suggesting their importance to the structure of FGL2. Four cysteines in the linear coiled-coil domain, linearly arranged as two pairs in a “Cys-X-X-Cys” motif, are critical for FGL2 oligomerization.

Transcription of the human fgl2 gene can produce 4 different mRNAs, 3 alternatively spliced variants, and 1 unspliced form. The detailed manner of cleavage leading to the function divergence between mFGL2 and sFGL2 remains unclear. A stretch of hydrophobic amino acids at the N-terminus of FGL2 served as signal peptide for sFGL2 secretion, but how sFGL2 was cleaved and secreted remains unknown. It has been reported that the C-terminal region of sFGL2 is critical for sFGL2-mediated immunoregulation [Chan, C. W. et al., J Immunol., 2003, 170:4036-4044].

sFGL2 in its natural state exists as an oligomer consisting of 4 monomers. Recently, it was shown that murine monomeric FGL2 has enhanced immunosuppressive activity in comparison to oligomeric FGL2. Moreover, all the functional motifs of FGL2 were shown to be located within the globular FRED domain. Further, monomeric FGL2 showed six to seven-fold lower binding affinity to murine bone marrow derived dendritic cells (BM-DCs) when compared with oligomeric FGL2 (Liu, H. el al., The international journal of biochemistry & cell biology, 2013, 45:408-418).

Several attempts have been made to use full-length mouse sFGL2 as an immunomodulator. Levy et al. (“USE OF SOLUBLE FGL2 AS AN IMMUNOSUPPRESSANT; WO2003/074068) describe using full-length mouse sFGL2 to inhibit xenogeneic T cell proliferation. Similarly, Chen et al. reported that full-length mouse sFGL2 inhibited T cell proliferation, and dendritic cell maturation, but had no inhibitory effects on cytotoxic T lymphocyte activity [Chan, C. W. et al., J Immunol., 2003, 170:4036-4044]. Immunosuppressant and immunomodulatory compositions are always greatly needed. Thus, a FGL2 composition that is effective on human cells, and can be used for treatment of autoimmune disease is of great interest.

### SUMMARY OF THE INVENTION

The present invention provides an immunomodulatory chimeric molecule comprising: a fibrinogen-related domain (FRED) from human fibrinogen-like protein 2 (FGL2), or an immunomodulatory fragment thereof, and human serum albumin (HSA). Pharmaceutical compositions comprising the chimeric molecule, and methods for reducing inflammation and treating autoimmune disease in a subject by administering the immunomodulatory pharmaceutical compositions are also provided.

According to a first aspect, there is provided an immunomodulatory chimeric molecule, the chimeric molecule comprising: a fibrinogen-related domain (FRED) from human fibrinogen-like protein 2 (FGL2), or an immunomodulatory fragment or analog thereof, and a half-life extending moiety.

According to another aspect, there is provided a pharmaceutical composition, comprising a therapeutically effective amount of a chimeric molecule of the invention.

According to another aspect, there is provided a method of reducing inflammation in a subject in need thereof, the method comprising administering to the subject a chimeric molecule of the invention or a pharmaceutical composition of the invention, thereby reducing inflammation in a subject.

According to another aspect, there is provided a method of treating an autoimmune disease in a subject in need thereof, the method comprising administering to the subject a chimeric molecule of the invention or a pharmaceutical composition of the invention, thereby treating an autoimmune disease in a subject.

According to some embodiments, the half-life extending moiety is selected from human serum albumin (HSA) and monomeric Fc.

According to some embodiments, the chimeric molecule is a stronger immunomodulator than the human FRED alone.

According to some embodiments, the immunomodulation is immunosuppression.

According to some embodiments, the immunomodulation comprises at least one of: reducing secretion of at least one inflammatory cytokine and reducing proliferation of an immune cell.

According to some embodiments, the immune cell is selected from a T cell, a B cell and a dendritic cell.

According to some embodiments, the half-life extending moiety is conjugated to the N- or C-terminus of the FRED or the immunomodulatory fragment or analog thereof.

According to some embodiments, the FRED or the immunomodulatory fragment or analog thereof and the half-life extending moiety are connected by a linker.

According to some embodiments, the linker is an amino acid linker.

According to some embodiments, the linker comprises the amino acid sequence GGGGS.

According to some embodiments, the linker comprises or consists of the amino acid sequence GGGGSGGGGSGGGGS (SEQ ID NO: 4).

According to some embodiments, the linker does not comprise a sequence of at least 10 amino acids from FGL2.

According to some embodiments, the FRED consists of the amino acid sequence provided in SEQ ID NO: 3.

According to some embodiments, the chimeric molecule of the invention further comprises a tag.

According to some embodiments, the tag is a His tag.

According to some embodiments, the His tag is a 6x His tag.

According to some embodiments, the tag is a C-terminal tag.

According to some embodiments, the pharmaceutical composition of the invention further comprises a pharmaceutically acceptable carrier, excipient or adjuvant.

According to some embodiments, reducing inflammation comprises reducing expression of at least one proinflammatory cytokine.

According to some embodiments, the proinflammatory cytokine is selected from interferon gamma (IFN-g), tumor necrosis factor alpha (TNFa) and interleukin 6 (IL-6).

According to some embodiments, the reducing expression is reducing expression by an immune cell selected from a T cell and a dendritic cell.

According to some embodiments, the reducing inflammation comprises reducing proliferation of an immune cell in the subject, the immune cell selected from a T cell and a dendritic cell.

According to some embodiments, the autoimmune disease is selected from the group consisting of: rheumatoid arthritis, inflammatory bowel disease, colitis, ulcerative colitis, autoimmune encephalomyelitis (EAE), lupus, Multiple Sclerosis (MS) and Crohn's disease.

According to some embodiments, the autoimmune disease is EAE or MS.

According to some embodiments, treating comprises reducing inflammation in the subject.

According to some embodiments, the reducing inflammation comprises reducing expression of at least one proinflammatory cytokine.

According to some embodiments, the proinflammatory cytokine is selected from interferon gamma (IFN-g), tumor necrosis factor alpha (TNFa) and interleukin 6 (IL-6).

According to some embodiments, the reducing expression is reducing expression by an immune cell selected from a T cell and a dendritic cell.

According to some embodiments, the treating comprises reducing proliferation of an immune cell in the subject, the immune cell selected from a T cell and a dendritic cell.

According to some embodiments, the treating comprises reducing differentiation of monocytes to mature dendritic cells.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B: Photographs of (IA) an SDS-PAGE gel showing recombinant His-tagged FRED and (1B) a western blot with anti-His primary antibody. Lane M1: Protein Marker, TaKaRa, Cat. No. 3452. Lane M2: Protein Marker, GenScript, Cat. No. M00521. Lane 1: Reducing condition. Lane 2: Non-reducing condition. Lane P: Multiple-tag as positive control.

FIGS. 2A-2C: Histograms of FACS analysis showing surface binding of FRED to (2A) activated T cells, (2B) immature dendritic cells and (2C) mature dendritic cells. Isotype controls are shown in black, FRED binding is shown in light grey.

FIGS. 3A-3D: (3A) A histogram showing the expression of CD25 on the surface of isolated, activated CD4 positive T cells. Isotype control is shown in black, CD25 binding in light grey. (3B) Dot plots of sorted CD25 positive and CD25 negative cells stained for intracellular FGL2 and FOXP3. (3C) Histogram of FGL2 intracellular expression in CD25 positive cells. Isotype control is shown in black, FGL2 binding in light grey. (3D) Histogram of surface FGL2 expression in CD25 positive cells. Isotype control is shown in black, FGL2 binding in light grey.

FIGS. 4A-4B. Bar charts showing (4A) proliferation percentage and (4B) IFN-g secretion from activated T cells with and without FRED treatment.

FIG. 5. A bar chart showing IFN-g secretion from activated dendritic cells with and without FRED treatment.

FIGS. 6A-6C. Bar charts showing (6A) proliferation, (6B) INF-g secretion, and (6C) IL-6 secretion from MOG reactive mouse splenocytes with and without FRED treatment. Stimulation with 20 μg/ml MOG peptide (left panels) and 1 μg/ml MOG peptide (right panels) is shown.

FIG. 7. A histogram showing FRED binding to MOG reactive mouse splenocytes. Isotype control is shown in black, FRED binding in light grey.

FIG. 8. A bar chart showing the proliferation index of T effector cells after a mixed leukocyte reaction assay with and without FRED. CLTA-4 was used as a positive control.

FIGS. 9A-9B. (9A) A line graph showing binding of H-FRED to naïve and activated T cells. (9B) A bar graph showing proliferation of naïve T cells with and without H-FRED treatment for 48 and 96 hours.

FIGS. 10A-10B. Bar charts showing (10A) proliferation and (10B) IFN-g secretion from activated T cells with and without H-FRED or FRED-H treatment.

FIGS. 11A-11B. (11A) Dot plots of naïve T cells before and after activation with and without treatment with H-FRED. (11B) A table summarizing the effect of H-FRED on naïve T cell activation.

FIGS. 12A-12B. (12A) Line graph of T cell clusters per image taken with an IncuCyte live cell analysis system at various time points of naïve T cells, activated T cells incubated with a control HSA peptide and activated T cells incubated with H-FRED. (12B) Micrographs of T cell clusters from the experiment in 12A.

FIGS. 13A-13C. (13A) Dot plots of CD80 and CD83 expressing cells after monocyte differentiation to mature dendritic cells (DCs) in the absence or presence of HSA control peptide, H-FRED, Fc control peptide, and monoFc-FRED. (13B-13C) Bar charts measuring secretion of pro-inflammatory cytokines (13B) TNFa and (13C) IL-6 from the naïve and differentiated DCs with and without the various peptides.

FIGS. 14A-14C. (14A) A bar chart showing proliferation of activated T cells with and without H-FRED, His-H-FRED and His-FRED-H. (14B-14C) Bar charts showing IFN-g secretion from activated T cells with and without FRED-His and (14B) FRED-Fc, or (14C) His-FRED-monoFc and His-monoFc-FRED.

FIGS. 15A-15B. Bar charts showing (15A) cell proliferation and (15B) IFN-g secretion from co-incubated MS patient T cells and B-cells with and without addition of two concentration of myelin basic protein and with and without HSA control peptide, H-FRED, Fc control peptide and monoFc FRED.

### DETAILED DESCRIPTION OF THE INVENTION

The present invention, in some embodiments, provides an immunomodulatory chimeric molecule, the chimeric molecule comprising: a fibrinogen-related domain (FRED) from human fibrinogen-like protein 2 (FGL2), or an immunomodulatory fragment or analog thereof, and human serum albumin (HSA) are provided. Pharmaceutical compositions comprising the molecule are also provided, as are methods for reducing inflammation and treating autoimmune disease in a subject by administering the immunomodulatory pharmaceutical compositions.

The invention is based, in part, on the surprising finding that human FRED alone, without the rest of the FGL2 molecule, is a potent immunomodulator that can reduce inflammation in vitro and in vivo. The invention is further based, on the unexpected superiority of chimeric molecules comprising additional stabilizing and targeting moieties. In particular, the addition of HSA was found to significantly enhance the immunomodulatory properties of the FRED.

By a first aspect, there is provided an immunomodulatory molecule, the molecule comprising or consisting of a fibrinogen-related domain (FRED) from human fibrinogen-like protein 2 (FGL2), or an immunomodulatory fragment or analog thereof.

In some embodiments, human FGL2 consists of the amino acid sequence

(SEQ ID NO: 1)
MKLANWYWLSSAVLATYGFLVVANNETEEIKDERAKDVCPVRLESRGKC
EEAGECPYQVSLPPLTIQLPKQFSRIEEVFKEVQNLKEIVNSLKKSCQD
CKLQADDNGDPGRNGLLLPSTGAPGEVGDNRVRELESEVNKLSSELKNA
KEEINVLHGRLEKLNLVNMNNIENYVDSKVANLTFVVNSLDGKCSKCPS
QEQIQSRPVQHLIYKDCSDYYAIGKRSSETYRVTPDPKNSSFEVYCDME
TMGGGWTVLQARLDGSTNFTRTWQDYKAGFGNLRREFWLGNDKIHLLTK
SKEMILRIDLEDENGVELYALYDQFYVANEFLKYRLHVGNYNGTAGDAL
RENKHYNHDLKFFTTPDKDNDRYPSGNCGLYYSSGWWFDACLSANLNGK
YYHQKYRGVRNGIFWGTWPGVSEAHPGGYKSSFKEAKMMIRPKHFKP.

In some embodiments, human FGL2 lacks the signal peptide and consists or comprises the amino acid sequence

(SEQ ID NO: 2)
NNETEEIKDERAKDVCPVRLESRGKCEEAGECPYQVSLPPLTIQLPKQF
SRIEEVFKEVQNLKEIVNSLKKSCQDCKLQADDNGDPGRNGLLLPSTGA
PGEVGDNRVRELESEVNKLSSELKNAKEEINVLHGRLEKLNLVNMNNIE
NYVDSKVANLTFVVNSLDGKCSKCPSQEQIQSRPVQHLIYKDCSDYYAI
GKRSSETYRVTPDPKNSSFEVYCDMETMGGGWTVLQARLDGSTNFTRTW
QDYKAGFGNLRREFWLGNDKIHLLTKSKEMILRIDLEDFNGVELYALYD
QFYVANEFLKYRLHVGNYNGTAGDALRFNKHYNHDLKFFTTPDKDNDRY
PSGNCGLYYSSGWWFDACLSANLNGKYYHQKYRGVRNGIFWGTWPGVSE
AHPGGYKSSFKEAKMMIRPKHFKP.

In some embodiments, the FRED domain is the most C-terminal domain of FGL2. In some embodiments, the FRED domain is a C-terminal globular domain. In some embodiments, the FRED domain does not comprise a coiled-coil domain. In some embodiments, the FRED domain is not a FGL2 oligomerization domain. In some embodiments, the FRED molecule of the invention does not dimerize or oligomerize. In some embodiments, the FRED domain is human FRED. In some embodiments, the FRED domain is not mouse or murine FRED. In some embodiments, the FRED domain consists of amino acids 204-439 of human FGL2. In some embodiments, the FRED domain consists of amino acids 204-439 of SEQ ID NO: 1. In some embodiments, the FRED domain consists of the amino acid sequence

(SEQ ID NO: 3)
PVQHLIYKDCSDYYAIGKRSSETYRVTPDPKNSSFEVYCDMETMGGGWT
VLQARLDGSTNFTRTWQDYKAGFGNLRREFWLGNDKIHLLTKSKEMILR
IDLEDENGVELYALYDQFYVANEFLKYRLHVGNYNGTAGDALRENKHYN
HDLKFFTTPDKDNDRYPSGNCGLYYSSGWWFDACLSANLNGKYYHQKYR
GVRNGIFWGTWPGVSEAHPGGYKSSFKEAKMMIRPKHFKP.

In some embodiments, the molecule comprises a fibrinogen-related domain (FRED) from human fibrinogen-like protein 2 (FGL2) or an immunomodulatory fragment or analog thereof. Testing immunomodulation can be performed using any known assay. Such assays include, but are not limited to, cytokine panels/measuring following stimulation of T cells, T cell proliferation assays, mixed leukocyte reactions (MLR) assay, and macrophage maturation assays. In some embodiments, the fragment comprises at least 50, 75, 100, 125, 150, 175, 200, 225 or 230 amino acids of the FRED. Each possibility represents a separate embodiment of the invention.

As used herein, the term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention. An analog is further defined as a polypeptide that is similar, but not identical, to the molecule of the invention and that is still immunomodulatory in the way that that human FRED is immunomodulatory. An analog, may have deletions or mutations that result in an amino acids sequence that is different than the amino acid sequence of the molecule of the invention. It should be understood, that all analogs of the molecule of the invention would still be immunomodulatory. Further, an analog may be analogous to a fragment of the molecule of the invention, however, in such a case the fragment must comprise at least 50 consecutive amino acids of the molecule of the invention. An analog is not a FRED from another species. In some embodiments, the analog is not murine FRED. In some embodiments, the analog is not mouse FRED.

In some embodiments, an analog to the molecule of the invention comprises an amino acid sequence with at least 60%, at least 65%, at least 70%, at least 75%, at least 80%, at least 85%, at least 90%, at least 95%, at least 99% homology to the amino acid sequence presented in SEQ ID NO: 3. In some embodiments, an analog of the molecule of the invention comprises an amino acid sequence with at least 90% homology to the amino acid sequence presented in SEQ ID NO: 3.

In some embodiments, the FRED, or analog or fragment thereof, comprises a mutation that enhances immunomodulation. In some embodiments, the mutation increases FRED binding to a receptor. In some embodiments, the mutation increases binding to a Fcgamma receptor. In some embodiments, the mutation is in a Fcgamma binding domain. In some embodiments, the mutation decreases inflammation. In some embodiments, the mutation increases immunosuppression.

In some embodiments, the immunomodulation is immunosuppression. In some embodiments, the immunomodulation comprises reducing inflammation. In some embodiments, the reducing inflammation comprises decreasing expression of at least one proinflammatory cytokine. Cytokines are small protein molecules well known in the art. Examples of cytokines include macrophage derived chemokines, macrophage inflammatory proteins, interleukins, tumor necrosis factors. Non-limiting examples of proinflammatory cytokines include IL-1, IL-1B, IL-2, IL-6, IL-17, IFN-gamma, and TNF-alpha. In some embodiments, the proinflammatory cytokine is selected from interferon gamma (IFN-g) and interleukin 6 (IL-6).

In some embodiments, the immunomodulation comprises reducing proliferation of an immune cell. In some embodiments, the immune cell is selected from a T cell and a dendritic cell (DC). In some embodiments, the T cell is selected from a T effector cell and a cytotoxic T cell. In some embodiments, the immunomodulation comprises reducing proliferation of a T cell. In some embodiments, the immunomodulation comprises reducing proliferation of a DC. In some embodiments, the immunomodulation comprises reducing proliferation of a T cell or a DC. In some embodiments, the immunomodulation comprises reducing proliferation of a T cell and a DC. In some embodiments, the immunomodulation comprises at least one of reducing inflammation and reducing proliferation of an immune cell. In some embodiments, the immunomodulation comprises at least one of reducing secretion of at least one inflammatory cytokine and reducing proliferation of an immune cell. In some embodiments, the immunomodulation comprises reducing differentiation of a monocyte to a mature dendritic cell (DC).

In some embodiments, the reducing is at least a 10%, 20%, 25%, 30%, 40%, 50%, 60%, 70%, 75%, 80%, 90%, 95%, 97%, or 99% reduction. Each possibility represents a separate embodiment of the invention. In some embodiments, the reducing is a reduction to levels in a healthy subject. In some embodiments, the reducing is a reduction to non-pathological levels.

In some embodiments, the molecule of the invention does not dimerize. In some embodiments, the molecule of the invention does not oligomerize. In some embodiments, the molecule of the invention binds its target receptor as a monomer.

In some embodiments, the molecule further comprises at least one tag. The tag may be any tagging molecule or moiety known in the art, including, but not limited to a fluorescent tag, a short peptide tag or a protein tag. Non-limiting examples of fluorescent tags include GFP tags, CFP tags, YFP tags, RFP tags, CY3 tags, CY5 tags, CY7 tags, fluorescein tags, and ethidium bromide tags. Non-limiting examples of peptide tags include Myc tag, His tags, FLAG tags, HA-tags, SBP tags, and glutathione tags. Non-limiting examples of protein tags include GST tags, BCCP tags, MBP tags, and protein A tags. In some embodiments, the tag is cleavable. In some embodiments, the tag is used during production of the molecule and removed or cleaved before administration to a subject. In some embodiments, the tag is used for protein purification. In some embodiments, the molecule comprises tandem tags. In some embodiments, the tandem tags are used for tandem affinity purification. In some embodiments, the molecule comprises more than one copy of a given tag. It is well known in the art that some tags can be used as repeated tags, such as 3X FLAG and 6X His.

In some embodiments, the tag is a C-terminal tag. In some embodiments, the tag is an N-terminal tag. In some embodiments, the tag is not an N-terminal tag. In some embodiments, the tag is not at a terminus. In some embodiments, the tag comprises at least one moiety. In some embodiments, the tag comprises more than one moiety. In some embodiments, the tag is directly conjugated. In some embodiments, the tag is conjugated by a linker.

In some embodiments, the tag is a His tag. In some embodiments, the His tag is a 6X His tag. In some embodiments, the His tag is directly conjugated. In some embodiments, the His tag is directly conjugated to the FRED. In some embodiments, the His tag is conjugated by a linker. In some embodiments, the His tag is conjugated to the FRED by a linker. In some embodiments, the His tag is conjugated to the C-terminus of the molecule. In some embodiments, the His tag is conjugated to the C-terminus of the FRED.

The term “moiety”, as used herein, relates to a part of a molecule that may include either whole functional groups or parts of functional groups as substructures. The term “moiety” further means part of a molecule that exhibits a particular set of chemical and/or pharmacologic characteristics which are similar to the corresponding molecule.

As used herein, the term “conjugated” refers to any form of joining or bonding such as can be performed in a protein. In some embodiments, the conjugating is by a covalent bond.

In some embodiments, the molecule is a chimeric molecule and further comprises a stabilizing moiety. In some embodiments, the chimeric molecule comprises at least one stabilizing moiety. In some embodiments, the stabilizing moiety increases the half-life of the chimeric molecule. In some embodiments, the stabilizing moiety is a half-life increasing moiety. In some embodiments, increased half-life is half-life in a subject. In some embodiments, the increased half-life is in solution. In some embodiments, the solution is blood or plasma. As used herein, a “stabilizing moiety” refers to any molecule, or part of a molecule, known in the art to increase the stability of another molecule to which it is conjugated. Examples of stabilizing moieties include serum albumin, the Fc domain from IgG, hydroxyethyl starch (HES), CTP, exending and polyethylene glycol (PEG). In some embodiments, the stabilizing moiety is albumin. In some embodiments, the stabilizing moiety is serum albumin. In some embodiments, the stabilizing moiety is human serum albumin (HSA). In some embodiments, the stabilizing moiety is mouse serum albumin. In some embodiments, the stabilizing moiety is Fc. In some embodiments, the stabilizing moiety is human serum albumin (HSA) or Fc. In some embodiments, the Fc comprises a mutation that decreases or abolishes binding to an Fc receptor. In some embodiments, the Fc comprises the mutation N297G. In some embodiments, the Fc is monomeric Fc. In some embodiments, the Fc comprises a mutation that decreases or abolishes dimerization. In some embodiments, the chimeric protein comprises HSA. In some embodiments, the HSA is mutated to alter its stability. In some embodiments, the HSA is mutated to alter the stability of the molecule. In some embodiments, the HSA is mutated to increase stability of the molecule. In some embodiments, the HSA is mutated to increase half-life of the molecule. In some embodiments, the HSA is mutated to decrease stability of the molecule. In some embodiments, the HSA is mutated to decrease half-life of the molecule. In some embodiments, the sequence of HSA comprises or consists of the amino acid sequence

(SEQ ID NO: 6)
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL.

In some embodiments, a stabilizing moiety comprises a polyethylene glycol (PEG) molecule. In some embodiments, the stabilizing moiety consists of PEG. In some embodiments, a stabilizing moiety comprises a plurality of PEG molecules. In some embodiments, the stabilizing moiety is PEG. In some embodiments, the stabilizing moiety is a PEG molecule. In some embodiments, the stabilizing moiety comprises PEG or a PEG molecule. In some embodiments, the PEG is linear PEG. In some embodiments, the PEG is chained PEG. In some embodiments, the PEG is chains of PEG. In some embodiments, the PEG is branched PEG. In some embodiments, the PEG comprises PEG methyl ether. In some embodiments, the PEG is PEG dimethyl ether.

In some embodiments, the PEG is low molecular weight PEG. In some embodiments, the PEG is high molecular weight PEG. In some embodiments, the PEG comprises a molecular weight of at least 100, 200, 300, 400, 500, 600, 700, 800, 900, 1000, 1500, 2000, 2500, 3000, 3500, 4000, 4500, 5000, 10000, 15000 or 20000 grams/mol. Each possibility represents a separate embodiment of the invention. In some embodiments, the PEG comprises a molecular weight of at most 500, 600, 700, 800, 900, 1000, 1500, 2000, 2500, 3000, 3500, 4000, 4500, 5000, 10000, 15000, 20000, 25000, 30000, 35000, 40000, 45000, or 50000 grams/mol. Each possibility represents a separate embodiment of the invention. In some embodiments, the PEG comprises a molecular weight of about 100, 200, 300, 400, 500, 600, 700, 800, 900, 1000, 1500, 2000, 2500, 3000, 3500, 4000, 4500, 5000, 10000, 15000, 2000, 2500, 3000, 3500, 4000, 4500, 5000, 10000, 15000, 20000, 25000, 30000, 35000, 40000, 45000, or 50000 grams/mol. Each possibility represents a separate embodiment of the invention.

In some embodiments, the PEG molecule or molecules is attached to the polypeptide at a carboxylic acid residue. In some embodiments, the PEG molecule or molecules is attached to the polypeptide at an aspartic acid residue. In some embodiments, the PEG molecule or molecules is attached to the polypeptide at a glutamic acid residue. In some embodiments, the PEG molecule or molecules is attached to the polypeptide at a lysine residue. In some embodiments, the PEG molecule or molecules is attached to the polypeptide at a cysteine residue. In some embodiments, the PEG molecule or molecules is attached to the polypeptide at an aspartic acid residue, a glutamic acid residue, a lysine residue or a cysteine residue. Each possibility represents a separate embodiment of the invention. In some embodiments, the PEG molecule or molecules is attached to a linker. In some embodiments, the PEG molecule or molecules is attached via a linker. As used herein, “PEGylation” is the process of both covalent and non-covalent attachment or amalgamation of PEG to molecules and macrostructures. Methods of PEGylation are well known in the art and are disclosed in for example U.S. Pat. No. 7,610,156, which is incorporated by reference herein.

In some embodiments, the stabilizing moiety is an HSA binding polypeptide. In some embodiments, the HSA binding polypeptide comprises a single domain antibody. In some embodiments, the HSA binding polypeptide is a single domain antibody. In some embodiments, the second moiety comprises a single domain antibody comprising or consisting of the sequence:

(SEQ ID NO: 5)
EVQLVESGGGLVQPGNSLRLSCAASGFTFSSFGMSWVRQAPGKGLEWVS
SISGSGSDTLYADSVKGRFTISRDNAKTTLYLQMNSLRPEDTAVYYCTI
GGSLSRSSQGTLVTVSSAAA.

In some embodiments, the single domain antibody binding HSA is Alb8.

In some embodiments, the molecule is a chimeric molecule and further comprises a targeting moiety. In some embodiments, the chimeric molecule comprises at least one targeting moiety. In some embodiments, the targeting moiety targets the molecule to sites of inflammation. In some embodiments, the targeting moiety targets the molecule to sites of inflammation in the subject. As used herein, a “targeting moiety” refers to any molecule, or part of a molecule, known in the art to home to specific sites or conditions in a subject. Examples of inflammation targeting moieties include folic acid, HSA, nanoparticles, antibody fragments to immune cells and immune cell ligands. In some embodiments, the inflammation targeting moiety is HSA.

In some embodiments, the moieties incorporated into the chimeric molecule do not bind Fcgamma receptors. In some embodiments, the moieties incorporated into the chimeric molecule do not compete with FGL2 for binding to receptors. In some embodiments, the moieties incorporated into the chimeric molecule do not compete with FRED for binding to receptors.

In some embodiments, the stabilizing moiety increases the immunomodulatory effect of the FRED. In some embodiments, the targeting moiety increases the immunomodulatory effect of the FRED. In some embodiments, the stabilizing moiety and/or the targeting moiety increases the immunomodulatory effect of the FRED. In some embodiments, the HSA increases the immunomodulatory effect of the FRED. In some embodiments, the chimeric molecule of the invention is a stronger immunomodulator than the FRED alone. In some embodiments, the chimeric molecule of the invention has at least one immunomodulatory effect that is stronger than the immunomodulatory effect of FRED alone. In some embodiments, FRED alone is human FRED alone. In some embodiments, FRED alone consists of the amino acid sequence provide in SEQ ID NO: 3. In some embodiments, FRED alone consists of the amino acid sequence provided in SEQ ID NO: 3 conjugated to a tag. In some embodiments, FRED alone consists of the amino acid sequence provide in SEQ ID NO: 3 or the amino acid sequence provide in SEQ ID NO: 3 conjugated to a tag. In some embodiments, the immunomodulatory effect is reducing inflammation. In some embodiments, the immunomodulatory effect is reducing proliferation of an immune cell. In some embodiments, the immunomodulatory effect is selected from reducing inflammation and reducing proliferation of an immune cell.

In some embodiments, the increase is at least a 10%, 20%, 25%, 30%, 40%, 50%, 60%, 70%, 75%, 80%, 90%, 95%, 100%, 150%, 200%, 250%, 300%, 350%, 400%, 450%, 500%, 600%, 700%, 800%, 900%, or 1000% increase. Each possibility represents a separate embodiment of the invention.

In some embodiments, the FRED and the moiety are conjugated. In some embodiments, the FRED and moiety are directly conjugated. In some embodiments, the FRED and moiety are connected by a linker. In some embodiments, the linker is a protein linker. In some embodiments, the linker is an amino acid linker. In some embodiments, the linker comprises at least 2, 4, 5, 6, 8, 10, or 12 amino acids. Each possibility represents a separate embodiment of the invention. In some embodiments, the linker is a dipeptide. In some embodiments, the linker consists of 2 amino acids. In some embodiments, the linker comprises at least 2 amino acids. In some embodiments, the linker is a single amino acid. In some embodiments, the linker comprises at least 12 amino acids. In some embodiments, the linker comprises at most 4, 6, 8, 10, 12, 14, 15, 16, 18, or 20 amino acids. Each possibility represents a separate embodiment of the invention. In some embodiments, the linker comprises at most 10 amino acids. In some embodiments, the linker comprises at most 20 amino acids. In some embodiments, the linker comprises between 2 and 20, 2 and 18, 2 and 16, 2 and 15, 2 and 14, 2 and 12, 4 and 20, 4 and 18, 4 and 16, 4 and 15, 4 and 14, 4 and 12, 5 and 20, 5 and 18, 5 and 16, 5 and 15, 5 and 14, 5 and 12, 6 and 20, 6 and 18, 6 and 16, 6 and 15, 6 and 14, 6 and 12, 8 and 20, 8 and 18, 8 and 16, 8 and 15, 8 and 14, 8 and 12, 10 and 20, 10 and 18, 10 and 16, 10 and 15, 10 and 14, 10 and 12, 12 and 20, 12 and 18, 12 and 16 and 12 and 15 or 12 and 14. Each possibility represents a separate embodiment of the invention. In some embodiments, the linker is 12 amino acids in length. In some embodiments, the linker consists of 12 amino acids.

In some embodiments, the linker is a flexible linker. In some embodiments, the linker is an artificial linker. In some embodiments, the linker does not consist of a naturally occurring sequence. In some embodiments, the linker does not consist of a sequence of FGL2. In some embodiments, some embodiments, the linker is devoid of at least 5, 7, 10, 15, 20, 25, 30, 40 or 50 amino acids of a naturally occurring sequence. Each possibility represents a separate embodiment of the invention. In some embodiments, the amino acids are consecutive amino acids from the sequence. In some embodiments, the linker is devoid of at least 10 amino acids from a naturally occurring sequence. In some embodiments, the naturally occurring sequence is a sequence of FGL2. In some embodiments, the FGL2 is human FGL2. Thus, it will be understood by a skilled artisan that while the linker can be an amino acids sequence it will not be a part of FGL2, and thus the full-length FGL2, or other truncations of FGL2, cannot be used as the FRED and a linker.

In some embodiments, the linker comprises the amino acid sequence GGGGS. In some embodiments, the linker comprises repeats of the amino acid sequence GGGGS. In some embodiments, there are at least 1, 2, 3, 4, or 5 repeats of the sequence GGGGS. In some embodiments, the linker consists or comprises the amino acid sequence GGGGSGGGGSGGGGS (SEQ ID NO: 4). In some embodiments, the linker consists of 3 repeats of the sequence GGGGS. In some embodiments, the linker is not a fragment or amino acid sequence from FGL2. In some embodiments, the linker is not an extension of the FRED domain into upstream amino acid sequence of FGL2. It will be understood by a skilled artisan that the linker is not simply further sequence from human or murine FGL2, but rather is an unrelated protein sequence. In some embodiments, the linker does not comprise a domain that can dimerize or oligomerize. In some embodiments, there is a linker between the stabilizing moiety and the FRED, but not between the His tag and another moiety. In some embodiments, there are linkers between the stabilizing moiety and the FRED and between the His tag and another moiety.

In some embodiments, there is at least one stabilizing moiety. In some embodiments, there is one stabilizing moiety. In some embodiments, the stabilizing moiety is conjugated to the N-terminus of the FRED. In some embodiments, the stabilizing moiety is conjugated to C-terminus of the FRED. In some embodiments, the stabilizing moiety is conjugated to the N- or C-terminus of the FRED. In some embodiments, the stabilizing moiety is directly conjugated to the FRED. In some embodiments, the stabilizing moiety is connected to the FRED by a linker. In some embodiments, the His tag is conjugated to C-terminus of the stabilizing moiety. In some embodiments, the stabilizing moiety is conjugated to the N-terminus of the FRED and the His tag is conjugated to the C-terminus of the FRED. In some embodiments, the stabilizing moiety is conjugated to the C-terminus of the FRED and the His tag is conjugated to the C-terminus of the stabilizing moiety. In some embodiments, the linker is an N-terminal linker. In some embodiments, the linker is a C-terminal linker.

By another aspect, there is provided a nucleic acid molecule which encodes any one of the protein molecules of the invention.

In some embodiments, the nucleic acid molecule is an expression vector. Expression vectors are well known in the art and comprise all elements necessary for expression of the protein of the invention in a cell. This may include, but is not limited to promoters, regulatory elements, and untranslated regions. Expression vectors may be for expression in mammalian cells or bacterial cells for example. Non-limiting examples of expression vectors include pcDNA, pTT5, pGEX, pMAL, pCMV and pSV. In some embodiments, the expression vector is a mammalian expression vector. In some embodiments, the expression vector is pCDNA3.1.

By another aspect, there is provided a pharmaceutical composition comprising the molecule of the invention. In some embodiments, the pharmaceutical composition comprises a therapeutically effective amount of the molecule of the invention. In some embodiments, the pharmaceutical composition further comprises a pharmaceutically acceptable carrier, excipient or adjuvant.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, glycols, such as propylene glycol, polyols such as glycerin, sorbitol, mannitol and polyethylene glycol; esters such as ethyl oleate and ethyl laurate, pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, stearic acid, magnesium stearate, calcium sulfate, polyols, pyrogen-free water, isotonic saline, phosphate buffer solutions, as well as other non-toxic pharmaceutically compatible substances used in other pharmaceutical formulations. Wetting agents and lubricants such as sodium lauryl sulfate, as well excipients, stabilizers, antioxidants, and preservatives may also be present. Any non-toxic, inert, and effective carrier may be used to formulate the compositions contemplated herein.

The carrier may comprise, in total, from about 0.1% to about 99.99999% by weight of the pharmaceutical compositions presented herein.

The term “therapeutically effective amount” refers to an amount of a drug effective to treat a disease or disorder in a mammal. In some embodiments, the mammal is a human. Further, the term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the pharmaceutical composition is an immunomodulatory composition. In some embodiments, the pharmaceutical composition is an immunosuppressive composition. In some embodiments, the pharmaceutical composition is an anti-inflammatory composition. In some embodiments, the pharmaceutical composition is for use in treating inflammation. In some embodiments, the pharmaceutical composition is for use in treating an autoimmune disease. In some embodiments, the pharmaceutical composition is for use in decreasing inflammation. In some embodiments, the pharmaceutical composition is for use in treating an allergy. In some embodiments, the pharmaceutical composition is for use in decreasing rejection of a graft or transplant. In some embodiments, the pharmaceutical composition is for use in decreasing an immune response.

By another aspect, there is provided a method of reducing inflammation in a subject in need thereof, the method comprising administering to the subject a chimeric molecule of the invention or a pharmaceutical composition of the invention.

By another aspect, there is provided a method of treating an autoimmune disease in a subject in need thereof, the method comprising administering to the subject a chimeric molecule of the invention or a pharmaceutical composition of the invention.

By another aspect, there is provided a method of reducing an immune response in a subject in need thereof, the method comprising administering to the subject a chimeric molecule of the invention or a pharmaceutical composition of the invention.

By another aspect, there is provided use of the chimeric molecules of the invention or the pharmaceutical compositions of the invention for reducing inflammation in a subject.

By another aspect, there is provided use of the chimeric molecules of the invention or the pharmaceutical compositions of the invention for treating an autoimmune disease in a subject.

By another aspect, there is provided use of the chimeric molecules of the invention or the pharmaceutical compositions of the invention for reducing an immune response in a subject.

As used herein, the terms “administering,” “administration,” and like terms refer to any method which, in sound medical practice, delivers a composition containing an active agent to a subject in such a manner as to provide a therapeutic effect. Suitable routes of administration can include oral, parenteral, subcutaneous, intravenous, intramuscular, or intraperitoneal administration of a therapeutically effective amount of a composition of the present subject matter to a patient in need thereof.

The dosage administered will be dependent upon the age, health, and weight of the recipient, kind of concurrent treatment, if any, frequency of treatment, and the nature of the effect desired.

In some embodiments, the treating comprises reducing inflammation in the subject. In some embodiments, reducing an immune response comprises reducing inflammation in the subject. In some embodiments, reducing inflammation comprises reducing expression and/or secretion of at least one cytokine. In some embodiments, the cytokine is a proinflammatory cytokine. In some embodiments, the reducing is by an immune cell. In some embodiments, the proinflammatory cytokine is selected from IFN-g, TNFa and IL-6. In some embodiments, the cytokine is IFN-g. In some embodiments, the cytokine is TNFa. In some embodiments, the cytokine is IL-6. In some embodiments, the reducing inflammation comprises reducing proliferation of an immune cell. In some embodiments, the immune cell is selected from a B cell, T cell and a DC. In some embodiments, the immune cell is a T cell. In some embodiments, the immune cell is a B cell. In some embodiments, the immune cell is a monocyte. In some embodiments, the immune cell is a dendritic cell. In some embodiments, the immune cell is in the subject. In some embodiments, treating comprises reducing differentiation of monocytes to mature dendritic cells. In some embodiments, reducing an immune response comprises reducing differentiation of monocytes to mature dendritic cells.

Autoimmune diseases are any disease or condition in which the immune system attacks cells of the subject. Non-limiting examples of autoimmune diseases include arthritis, type 1 insulin-dependent diabetes mellitus, adult respiratory distress syndrome, inflammatory bowel disease, colitis, ulcerative colitis, Crohn's disease, dermatitis, meningitis, thrombotic thrombocytopenic purpura, Sjögren's syndrome, encephalitis, uveitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, encephalomyelitis, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune hemolytic anemia, Hashimoto's thyroiditis, Grave's disease, habitual spontaneous abortions, Reynard's syndrome, glomerulonephritis, dermatomyositis, chronic active hepatitis, celiac disease, tissue specific autoimmunity, degenerative autoimmunity delayed hypersensitivities, multiple sclerosis (MS), autoimmune complications of AIDS, atrophic gastritis, ankylosing spondylitis and Addison's disease.

In some embodiments, the autoimmune disease is selected from the group consisting of rheumatoid arthritis, diabetes, inflammatory bowel disease, autoimmune encephalitis (EAE), autoimmune encephalomyelitis, multiple sclerosis, lupus, multiple sclerosis (MS) and Crohn's disease. In some embodiments, the autoimmune disease is selected from the group consisting of rheumatoid arthritis, inflammatory bowel disease, autoimmune encephalomyelitis, and lupus. In some embodiments, the autoimmune disease is autoimmune encephalomyelitis (EAE). In some embodiments, the autoimmune disease is multiple sclerosis (MS).

In some embodiments, reducing an immune response is immune suppression. In some embodiments, the molecule of the invention may be used in place of any known immunosuppressant. In some embodiments, the method of the invention is for performing immunosuppression on a subject. In some embodiments, the immune response is an allergy. In some embodiments, the immune response is graft-versus host disease. In some embodiments, the immune response is the response to a graft or transplant. In some embodiments, the methods of the invention reduce rejection of a graft or transplant to the subject. In some embodiments, the method comprises administering a molecule of the invention to a subject receiving a transplant or graft. In some embodiments, the method comprises administering a molecule of the invention to a subject at at least one time point selected from: before transplant, during transplant or after transplant. In some embodiments, the molecule of the invention or the method of the invention decreases the risk of at least one side effect associated with transplantation. In some embodiments, the side effect is selected from: bacterial infection, viral infection, neoplasia and cardiovascular disease.

In some embodiments, the methods of the invention further comprise administering another immunomodulatory drug. In some embodiments, the methods of the invention further comprise administering another immunosuppressant.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.”is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Recombinant Human FRED Generation in a Mammalian Expression System

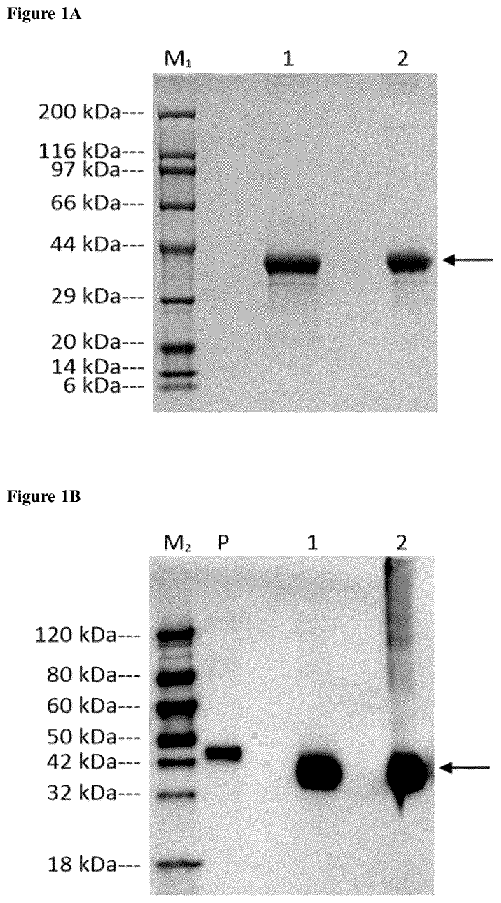

Recombinant human FRED protein (SEQ ID:1) with a His₆ tag at its C-terminus was successfully expressed in a mammalian expression system with the vector pTT5 in the host cell line CHO-3E7. Protein was obtained from the supernatant of cell culture and underwent a one-step purification using a HisTrap™ FF Crude column. SDS-PAGE followed by Coomassie blue staining of purified FRED protein showed a dominant band at approximately 40-KDa (FIG. 1A) which was confirmed by Western blotting (FIG. 1B). Purified protein was loaded under reducing conditions (Lane 1) and non-reducing conditions (Lane 2). Western blot analysis was probed using a Mouse-anti-His antibody.

### Example 2: FRED Binds to Human Activated T Cells and DC Cells

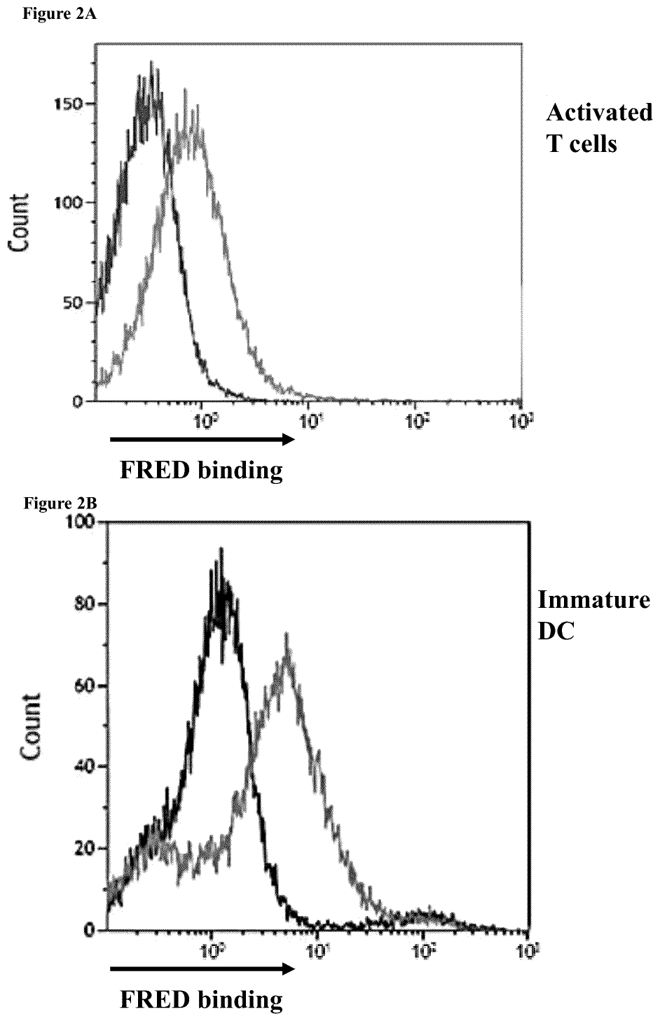

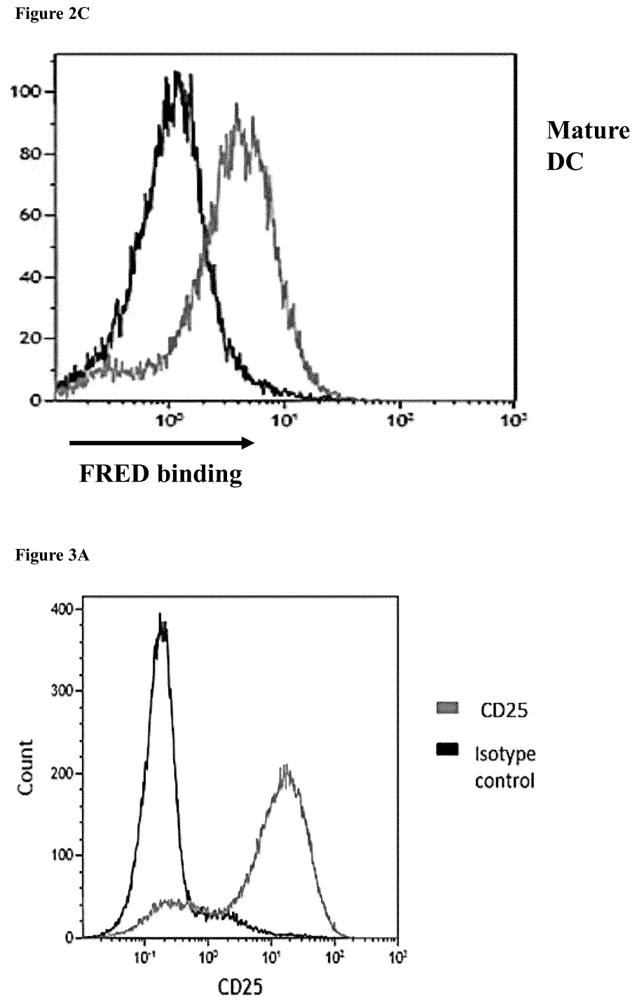

As can be seen in FIGS. 2A-2C, the recombinant human FRED successfully bound to activated human T cells, immature human dendritic cells (DC) and LPS-induced mature human DCs. An isolated CD3 positive population from blood samples of healthy donors was activated with anti-CD3 and anti-CD28 antibodies for five days. Following activation, the cells were incubated with FRED for 30 min followed by an anti-His-Tag APC-conjugated antibody and then analyzed by FACS. Human immature DC and LPS-induced mature DC cells were incubated with PE conjugated FRED for 30 min and then analyzed by FACS. These analyses demonstrate the ability of the recombinant FRED protein of the invention to bind to APC and T cells and that it does so similarly to sFGL2.

### Example 3: sFGL2 Expression in Human Regulatory T Cells

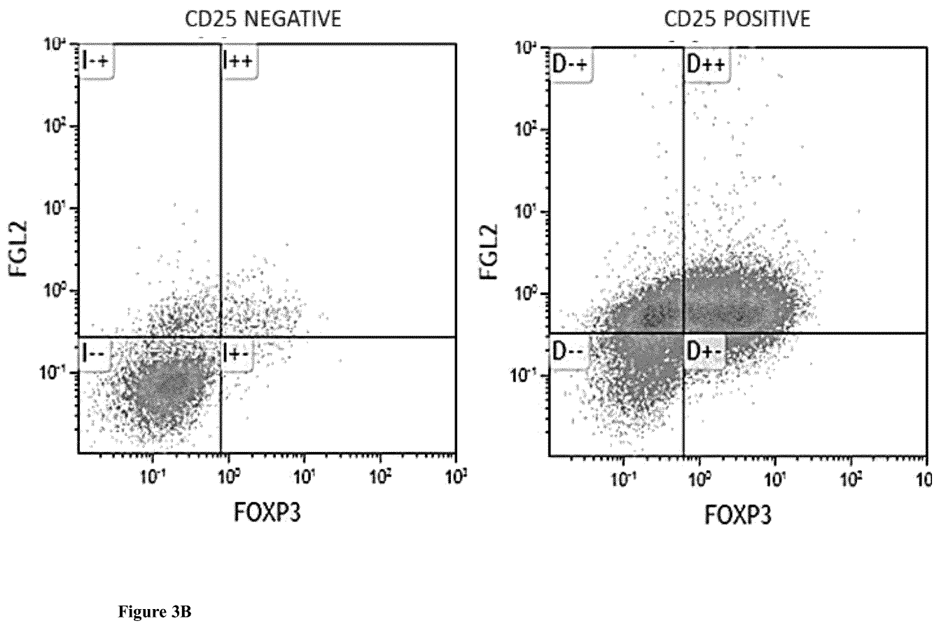

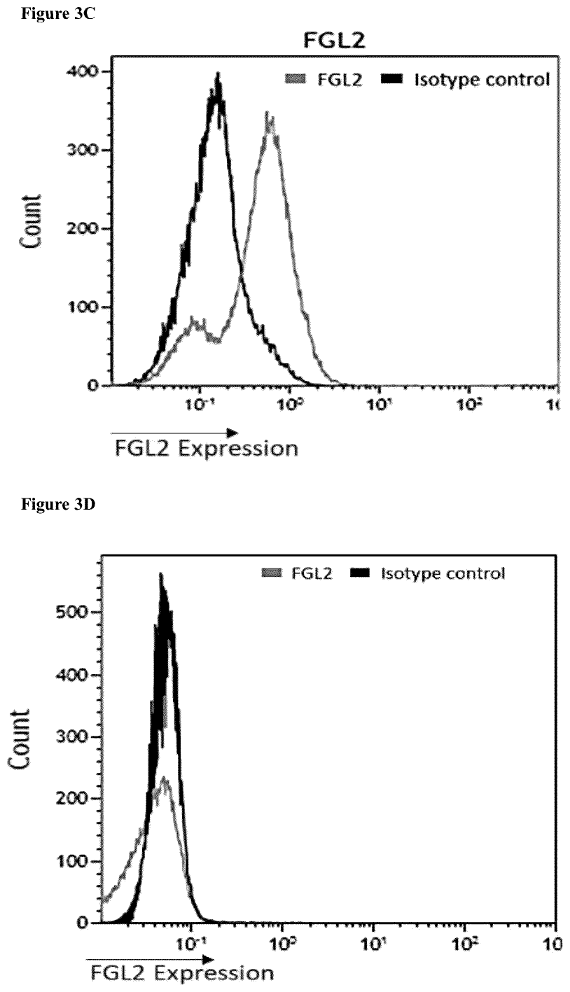

The inventors further evaluated the expression of sFGL2 in human regulatory T cells. Isolated CD4 positive T cells from blood samples of healthy donors were activated with CD3 and CD28 antibodies in the presence of IL-2 and TGF-β to obtain a regulatory T cell population (CD4⁺, CD25⁺ and FOXP3⁺). Golgi stop reaction was performed on the cells, followed by CD25 cell sorting by flow cytometry (FIG. 3A). Intracellular staining of FGL2 and FOXP3 was performed on both CD25 positive and negative cells (FIG. 3B). The staining results show that the FGL2 positive population is CD25 positive (FIG. 3C) or CD25 and FOXP3 positive (FIG. 3B). Moreover, membrane staining for FGL2 did not show any positive cells (FIG. 3D), suggesting that regulatory T cells exclusively express soluble FGL2 (sFGL2).

### Example 4: Inhibitory Effect of the Recombinant Human FRED Protein on T Cell Proliferation and IFN-Gamma Cytokine Secretion

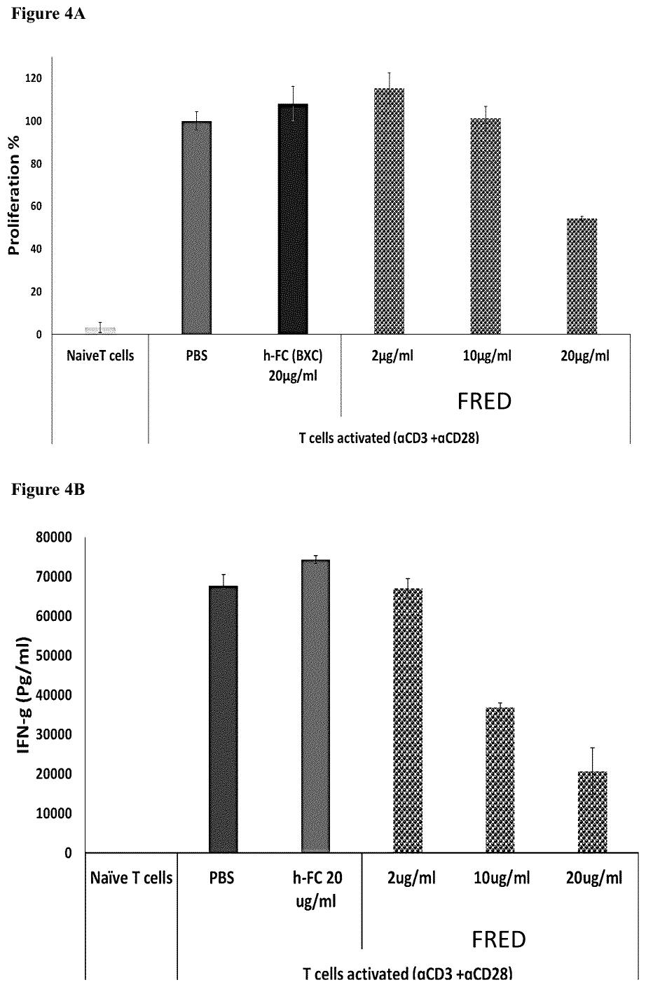

An isolated CD3 positive cell population from blood samples of healthy donors was activated with anti-CD3 and anti-CD28 antibodies for five days. His-tagged recombinant human FRED protein was added at three concentrations (2, 10 and 20 μg/mL) and human IgG (h-Fc) was added as negative control. Proliferation was determined with an MTT proliferation assay (FIG. 4A) and IFN-gamma was measured by a commercial ELISA assay (FIG. 4B). The results show the dose dependent inhibitory effect of His-tagged recombinant human FRED on activated T cell proliferation and cytotoxicity.

### Example 5: Recombinant Human FRED Protein Suppresses Activated Dendritic Cells

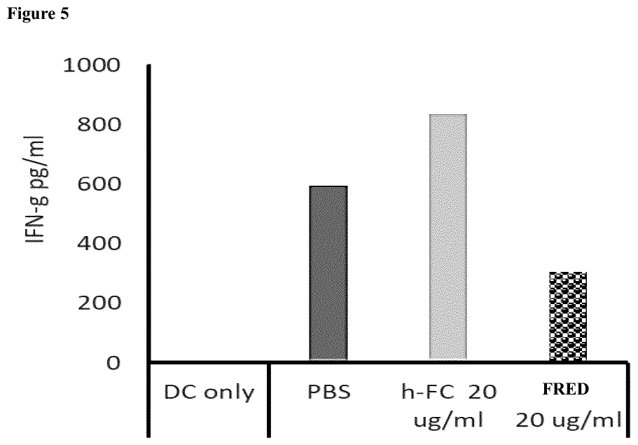

It has been suggested that FGL2 can bind to dendritic cells, inhibit their maturation process and ultimately mediate their conversion into immune suppressor cells. Induced immature dendritic cells (6 days of GM-CSF+IL4) were matured for an additional 2 days with LPS with and without adding His-tagged recombinant human FRED protein. Human IgG was added as negative control. IFN-gamma secretion was measured using a commercial ELISA assay. The results (FIG. 5) show that the recombinant His-tagged human FRED is able to suppress the secretion of IFN-gamma from DCs as it did for activated T cells.

### Example 6: Functionality of Recombinant Human FRED as an Immune-Regulator during MOG Peptide Stimulation of Specific MOG Reactive Mice Splenocytes

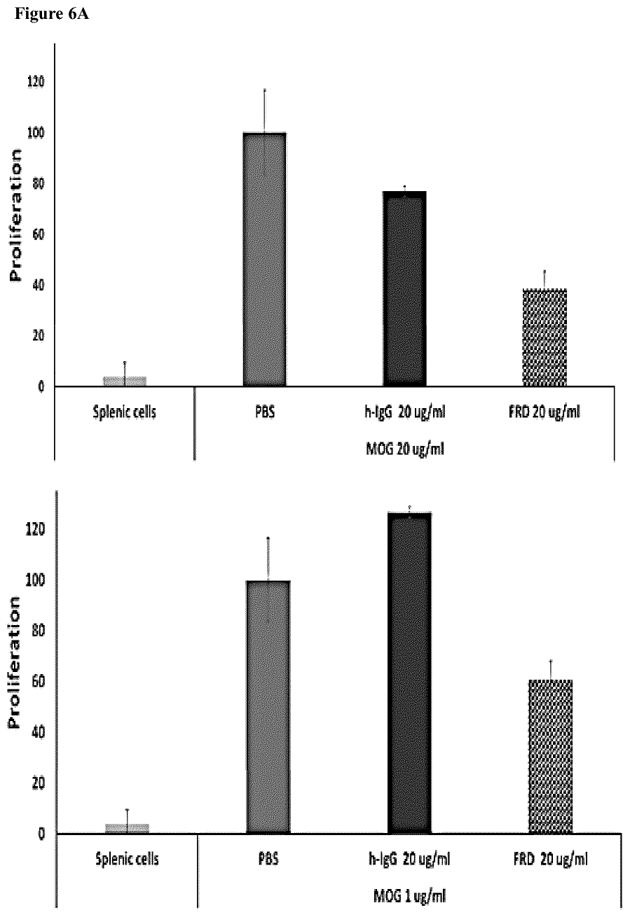

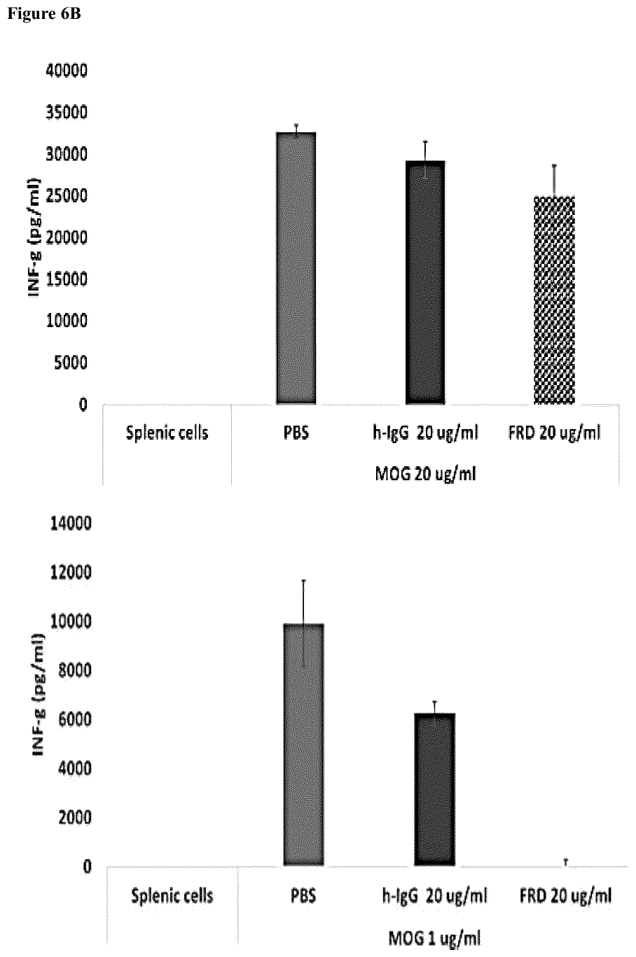

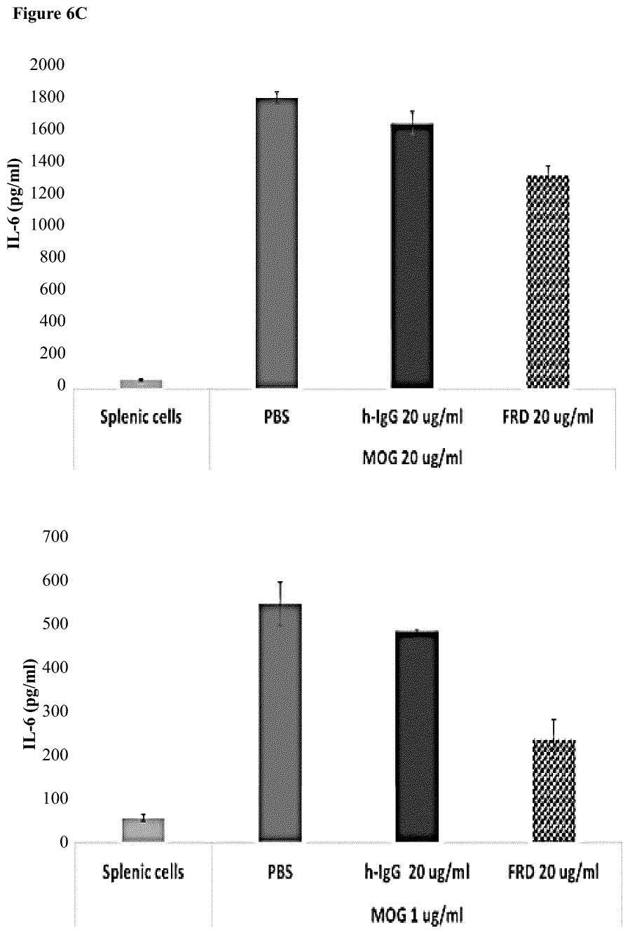

To examine the specific immune-modulatory effect of recombinant human FRED during the immune recognition of a specific antigen, an experimental autoimmune encephalomyelitis (EAE) model was used. Mice were injected with MOG₃₅₋₅₅ antigen for 9 days. After 9 days the mice splenocytes were harvested and cultured for 3 days with the MOG₃₅₋₅₅ peptide (20 or 1 μg/mL), to activate the MOG specific splenocytes, in the presence of recombinant human FRED protein (20 μg/mL) or human IgG as negative control. FIG. 6A shows cell proliferation results by MTT in the presence of 20 μg/mL (left) and 1 μg/mL (right) MOG peptide with and without FRED. FIGS. 6B and 6C show IFN-gamma and IL-6 secretion, respectively, following incubation with 20 μg/mL (left) and 1 μg/mL (right) of MOG peptide with and without recombinant human FRED. As can be seen from the results, the recombinant His-tagged human FRED successfully inhibits the proliferation of antigen-specific splenocytes and inhibits the secretion of proinflammatory cytokines by these cells.

### Example 7: Recombinant Human FRED Binds to MOG Reactive Mice Splenocytes

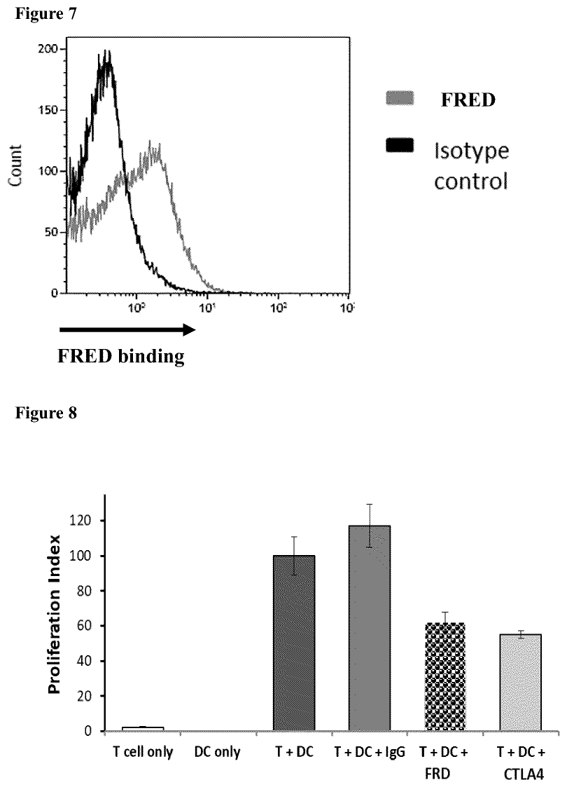

To ensure that the suppressive effect of recombinant human FRED on MOG reactive splenocytes is by binding of FRED to the splenocytes, mice were injected with MOG₃₅₋₅₅ antigen for 9 days. After 9 days the mice splenocytes were harvested and cultured with 20 μg/mL of MOG₃₅₋₅₅ peptide for 3 days. After 3 days the cells were incubated with PE conjugated recombinant human FRED for 30 minutes and analyzed by FACS. FIG. 7 shows that the recombinant His-tagged human FRED indeed binds to mice splenocytes.

### Example 8: Recombinant Human FRED Inhibits Effector T Cell Proliferation in an MLR Assay

DCs are unique antigen presenting cells, and their ability to induce proliferation of T cells in a mixed leukocyte reaction (MLR) assay is commonly used for the evaluation of their function. Therefore, the immune-suppressor effect of recombinant His-tagged human FRED on T cell proliferation in an MLR assay was tested. Immature human DCs were added to T cells from a different donor, in the absence or presence of recombinant human FRED for 4 days. Cytotoxic T lymphocyte associated antigen-4 (CTLA-4) was used as a positive control since it is a known mediator for immune inhibition by affecting DC/T cell interaction. Human IgG was added as a negative control. Cell proliferation was analyzed with a BRDU proliferation kit (FIG. 8). The results show that the recombinant His-tagged human FRED successfully inhibits proliferation of T effector cells following their interaction with DCs. Furthermore, this inhibition is as effective as that exhibited by CTLA-4.

### Example 9: Production of Different FGL2/FRED Constructs and Results

In order to generate a FRED construct with increased stability and half-life, multiple permutations of chimeric FRED proteins were designed and expression in different cell lines was attempted. Human serum albumin (HSA) and an Fc domain were added to FRED, full length FGL2 and monomeric FGL2 with a S91A mutation. The position of the HSA, Fc, His tag, and the length of the linker were all tested experimentally in different constructs. A summary of the various constructs is provided in Table 1.

TABLE 1

| | | Testable | Immune- |
|---|---|---|---|
| | Expression vector | protein | regulatory |
| Construct | and cell line | produced | function |
| FRED-His (FRED) | Expression Vector | Yes | Yes |
| | pTT5, CHO-3E7 | | |
| HSA-GGGGS₃-FRED- | pcDNA3.1(−), 293-6E | Yes | Yes |
| His (H-FRED) | | | |
| FRED-GGGGS₃-HSA- | pcDNA3.1(−), 293-6E | Yes | Yes |
| His (FRED-H) | | | |
| monoFc (N297G)- | Expression Vector | Mostly | N/A |
| GGS₂ + EK-FGL2 | pTT5, CHO-3E7 | aggregated | |
| (FL) | | form | |
| monoFc (N297G)- | CHO-3E7 | Yes | No |
| GGS₂-FRED | | | |
| Fc (N297G)-FGL2 | CHO-3E7 | Mostly | N/A |
| (23-439) | | aggregated | |
| | | form | |
| His-FRED-Fc | CHO-3E7/293 | Insignificant | N/A |
| (LALA + N297G) | | amounts of | |
| | | monomer | |
| His-FRED-Fc (Hinge | CHO-3E7/293 | No | N/A |
| mut + P238S) | | | |
| His-FRED-GGGS₂- | CHO-3E7/293 | Yes | No |
| monoFc (N297G) | | | |
| His-monoFc (N297G)- | CHO-3E7/293 | Yes | Yes |
| GGGS₂-FRED | | | |
| His-FRED-GGGGS- | CHO-S | Yes | No |
| HSA | | | |
| His-HSA-GGGGS- | CHO-S | Yes | No |
| FRED | | | |
| His-FGL2 | CHO-3E7/293 | No | N/A |
| (monomeric S91A) | | | |
| His-Fc-FGL2 | CHO-3E7/293 | No | N/A |
| (monomeric S91A) | | | |

### Example 10: H-FRED Binds to Human Naïve and Activated T Cells

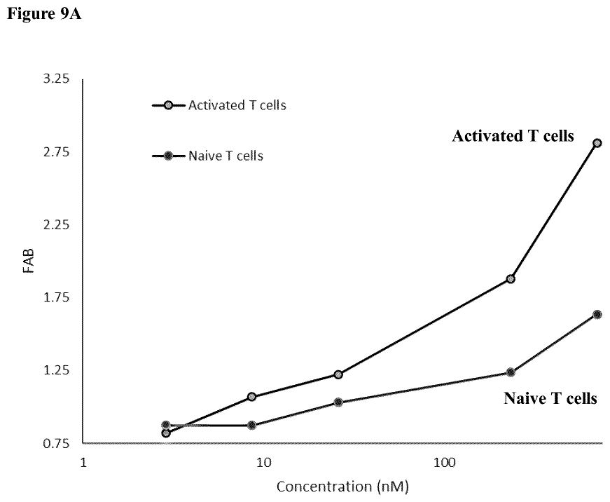

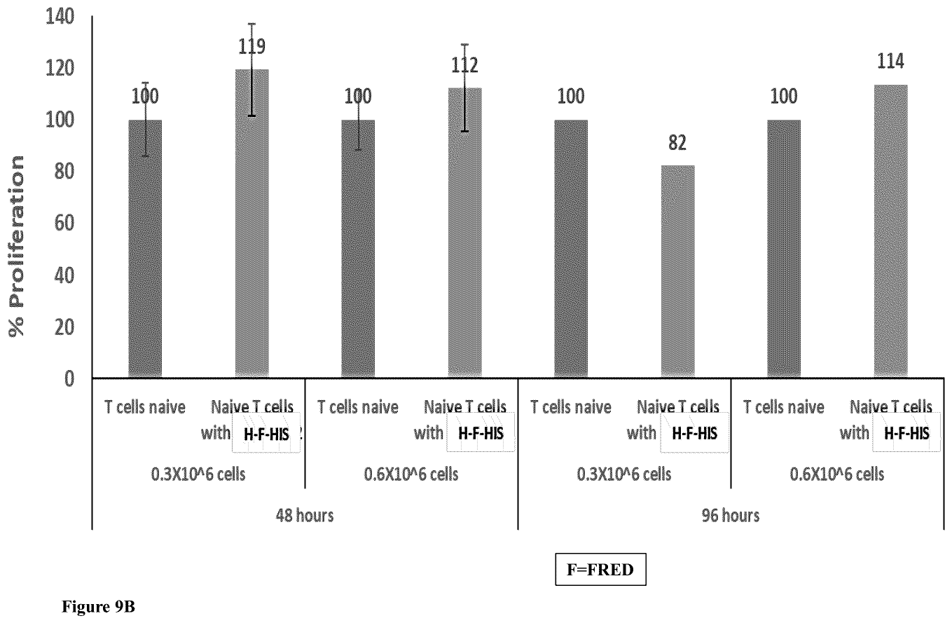

As can be seen in FIG. 9A, the recombinant HSA-FRED-His (H-FRED) successfully bound to human activated T cells, and with lower magnitude to naïve T cells. An isolated CD3 positive population from blood samples of healthy donors was incubated with or without activation by anti-CD3 and anti-CD28 antibodies for five days. On day 5 the cells were incubated with H-FRED (in 5 concentrations: 700, 233, 78, 26, 3) for 40 min followed by incubation with an anti-HSA PE-conjugated antibody and analysis by FACS. These analyses demonstrated the ability of the recombinant H-FRED protein to bind to naïve and activated T cells and that it does so similarly to sFGL2.

### Example 11: H-FRED does not Impair Viability of Naïve T Cells

An isolated CD3 positive population from blood samples of healthy donors was incubated for 48 and 96 hours with or without H-FRED (700 nM). Proliferation was determined with an MTT proliferation assay (FIG. 9B). The results show that H-FRED did not impair the viability of the naïve T cells even during the extended incubation. Further, the initial concentration of T cells did not impact viability.

### Example 12: Inhibitory Effect of H-FRED and FRED-H Proteins on T Cell Proliferation and IFN-Gamma Cytokine Secretion

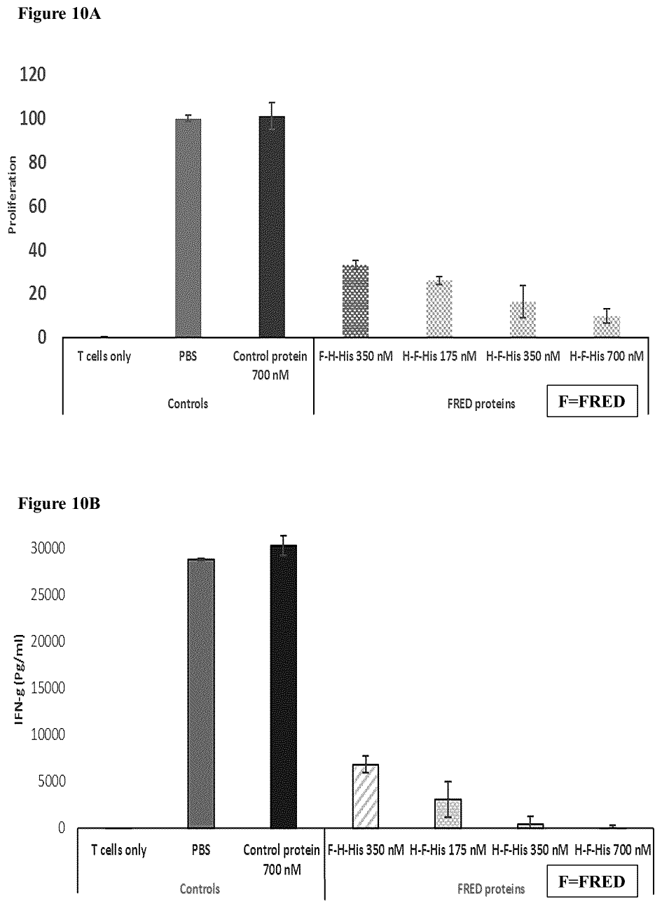

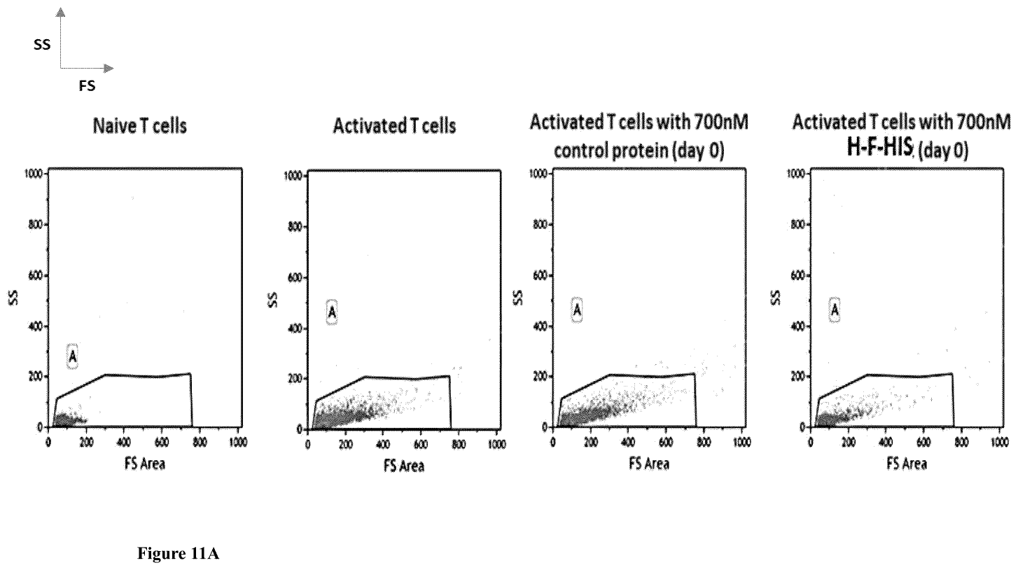

An isolated CD3 positive population from blood samples of healthy donors was activated with anti-CD3 and anti-CD28 antibodies for five days. Recombinant H-FRED (HSA-GGGGS₃-FRED-His) protein was added at three concentrations (175, 350 and 700 nM), FRED-H (FRED-GGGGS₃-HSA-His) was added at 350 nM and an irrelevant human protein (700 nM) fused to a His₆ tag was added as a negative control (FIG. 10A-10B). Proliferation was determined with an MTT proliferation assay (FIG. 10A) and IFN-gamma was measured by commercial ELISA assay (FIG. 10B). The results show the inhibitory effect of recombinant H-FRED and FRED-H proteins on activated T cells. Further, it appears that H-FRED was superior to FRED-H, at least for inhibition of IFN-g secretion in this context.

As 20 μg/ml FRED-His is equivalent to 700 nM, a comparison can be made between the immunomodulatory ability of FRED-His (FIG. 4A-4B) and that of H-FRED and FRED-H (FIG. 10A-10B). FRED-His was capable of inducing a 50% reduction in proliferation (FIG. 4A), while equimolar amounts H-FRED induced approximately a 90% reduction (FIG. 10A). Even at half the concentration (350 nM) both H-FRED and FRED-H were superior at inhibiting proliferation, and H-FRED was superior at even a quarter the concentration (175 nM). Similar results were observed for IFN-g secretion. FRED-His produced an approximately 70% reduction in IFN-g secretion (FIG. 4B), while equimolar amounts of H-FRED induced a greater than 95% reduction (FIG. 10B). At half, or even a quarter, the concentration, H-FRED was still superior, while FRED-H at half the concentration was roughly as effective as FRED-His. These results demonstrate the suppressing superiority of FRED/HSA chimeric proteins to FRED alone.

### Example 13: Evaluation of the Regulatory Effect of Recombinant H-FRED on T Cell Activity at Different Time Points from Activation

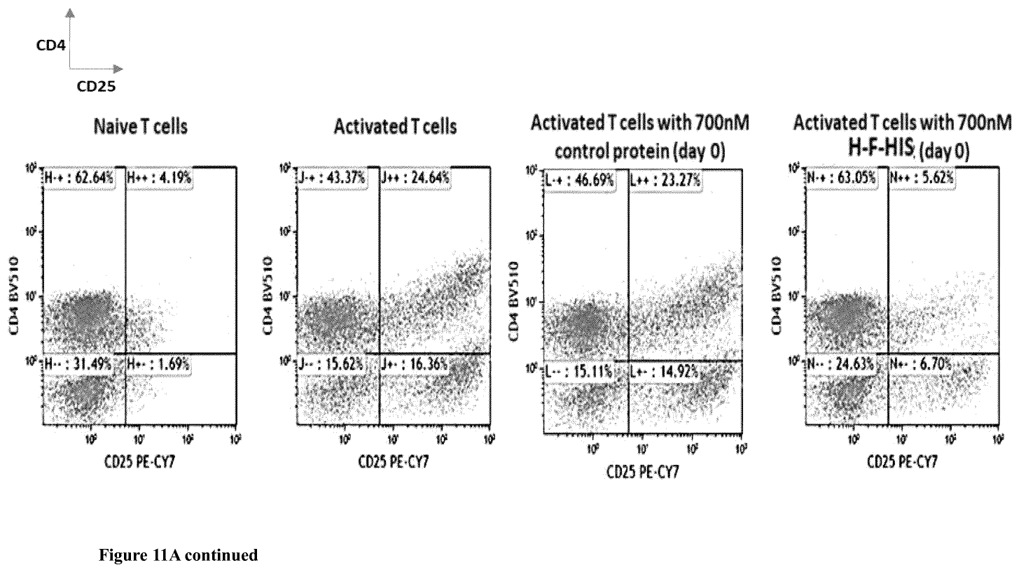

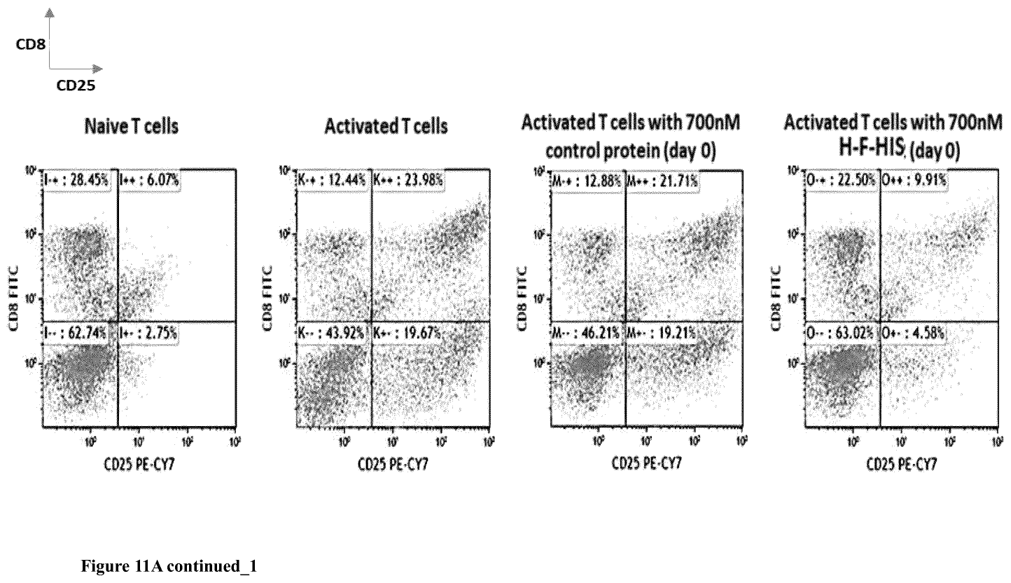

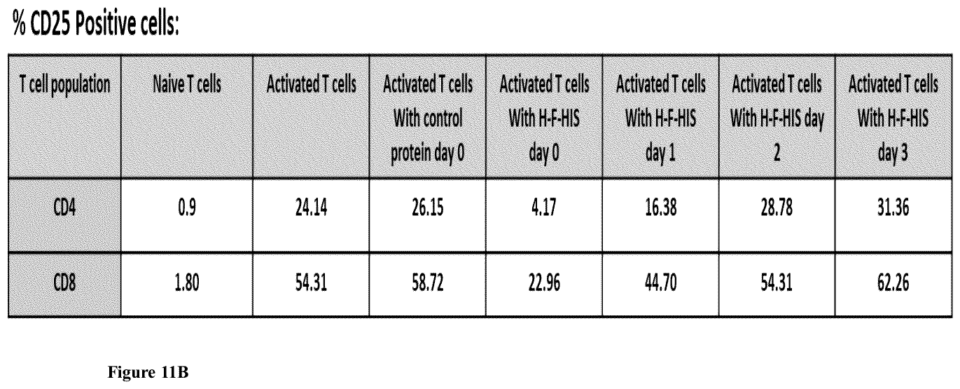

An isolated CD3 positive population from blood samples of healthy donors was stimulated with anti-CD3 and anti-CD28 antibodies for five days. Recombinant H-FRED and recombinant HSA-His control protein were added at a concentration of 700 nM at four different time points: at activation (0 days), 1 day after activation, 2 days after and 3 days after. At 5 days after activation the cells were stained with anti-CD4, anti-CD8 and anti-CD25 (as a marker for activation) and analyzed by FACS (FIG. 11A). The results show a significant inhibitory effect by H-FRED on the cells when the protein was added at the time of stimulation, with the treated cells resembling naïve T cells when compared to activated cells or to cells that received the control protein. As can be seen in FIG. 11B, the CD25 positive population (% CD25), which represents activated cells, is gradually elevated with each passing day from the stimulation until H-FRED treatment. This is true for both the CD4 and CD8 populations and shows that H-FRED prevents naïve T cells from becoming activated.

### Example 14: Inhibitory Effect of H-FRED on T Cell Clustering

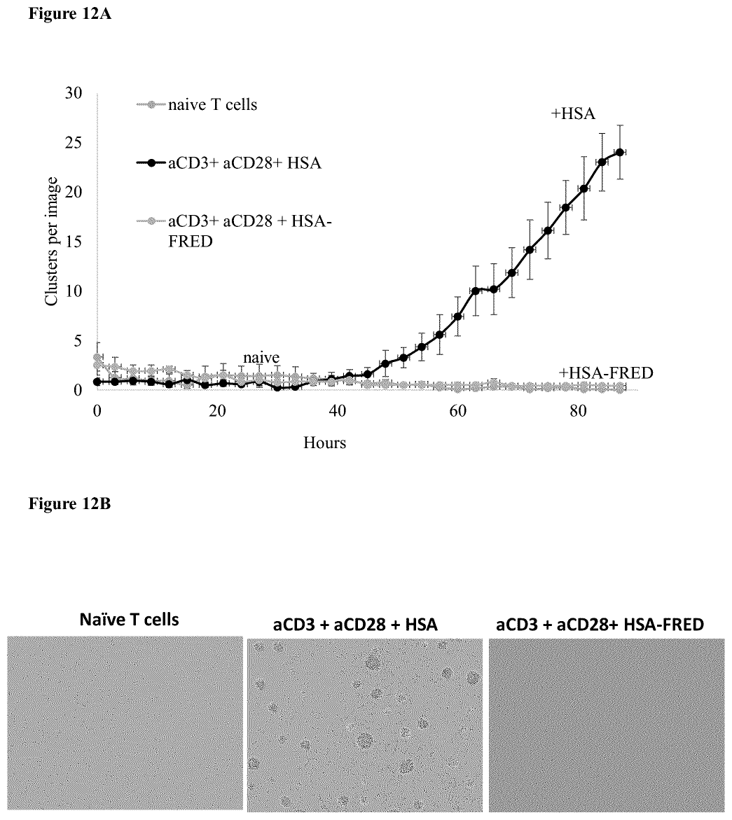

An isolated CD3 positive population from blood samples of healthy donors was stimulated with anti-CD3 and anti-CD28 antibodies for five days (a control population of naïve T cells was left unstimulated). Recombinant H-FRED (700 nM) and a control HSA peptide were added at day 0 and the formation of clusters was evaluated in real-time using an IncuCyte live cell analysis system (FIG. 12A). Starting after 40 hours clusters of T cells began appearing in the HSA treated cells, however, no clusters appeared in the H-FRED treated cells, which appeared comparable to the naïve (unstimulated) T cells (FIG. 12B). This demonstrates the ability of H-FRED to effectively inhibit T cell clustering.

### Example 15: H-FRED and Fc-FRED Inhibit Differentiation of Monocytes into Mature Dendritic Cells

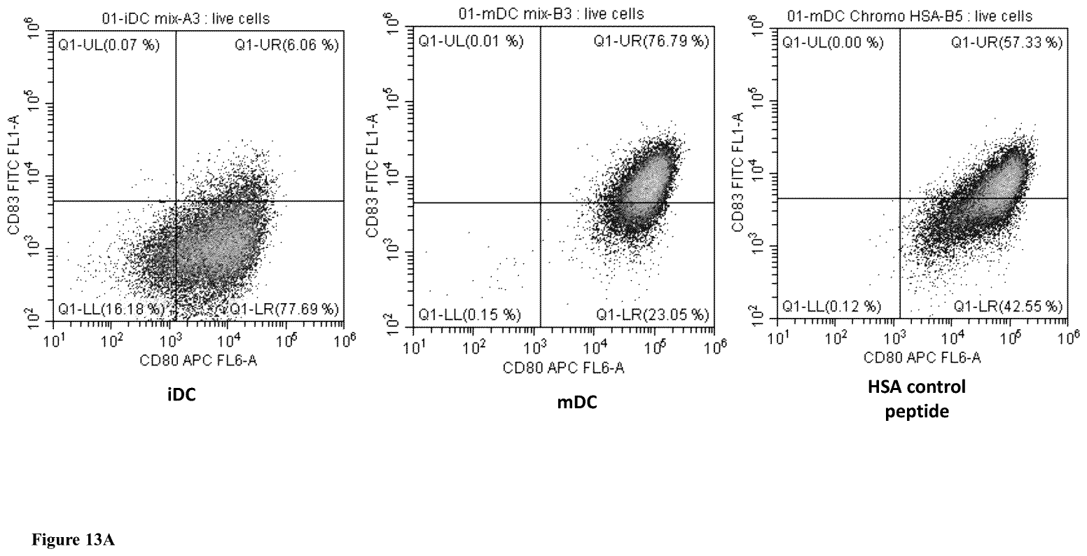

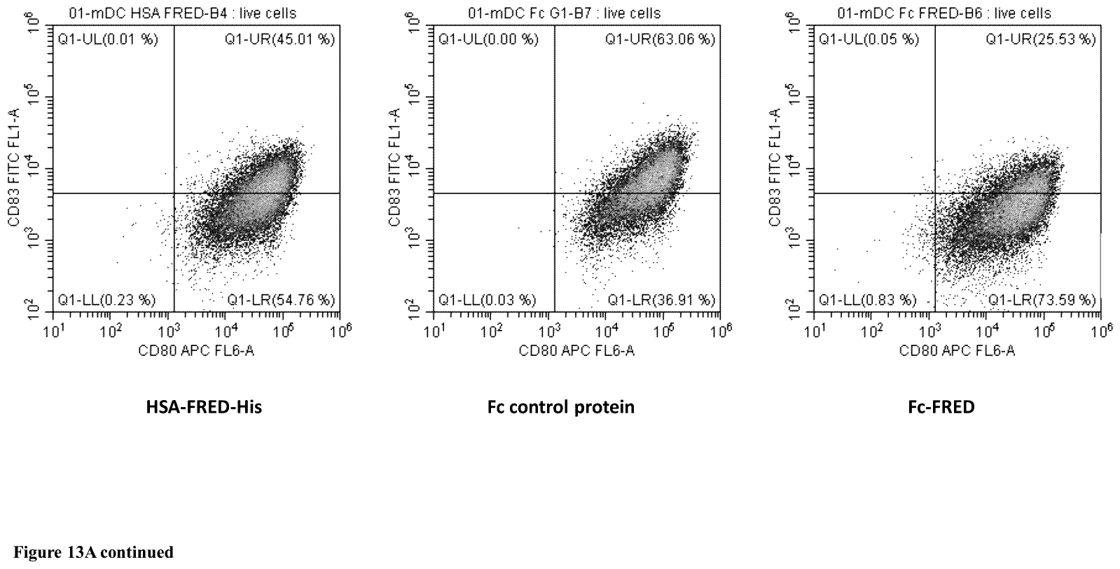

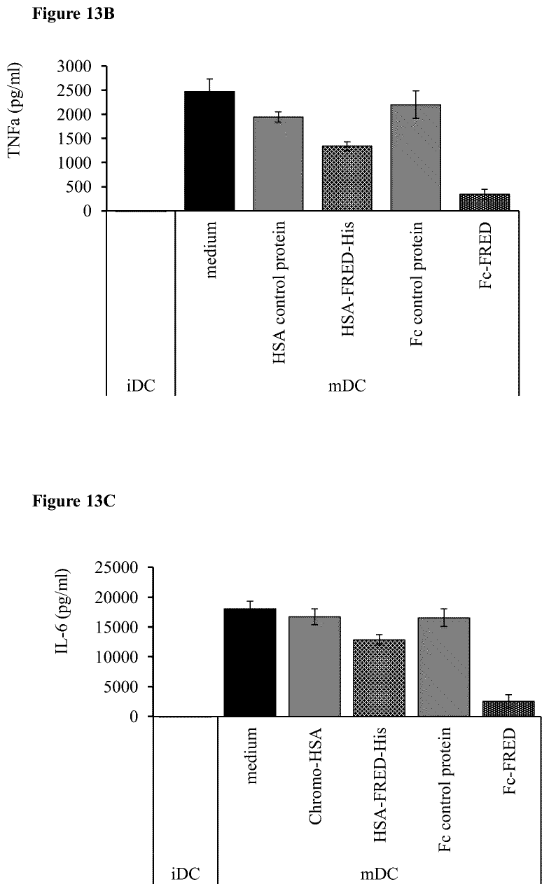

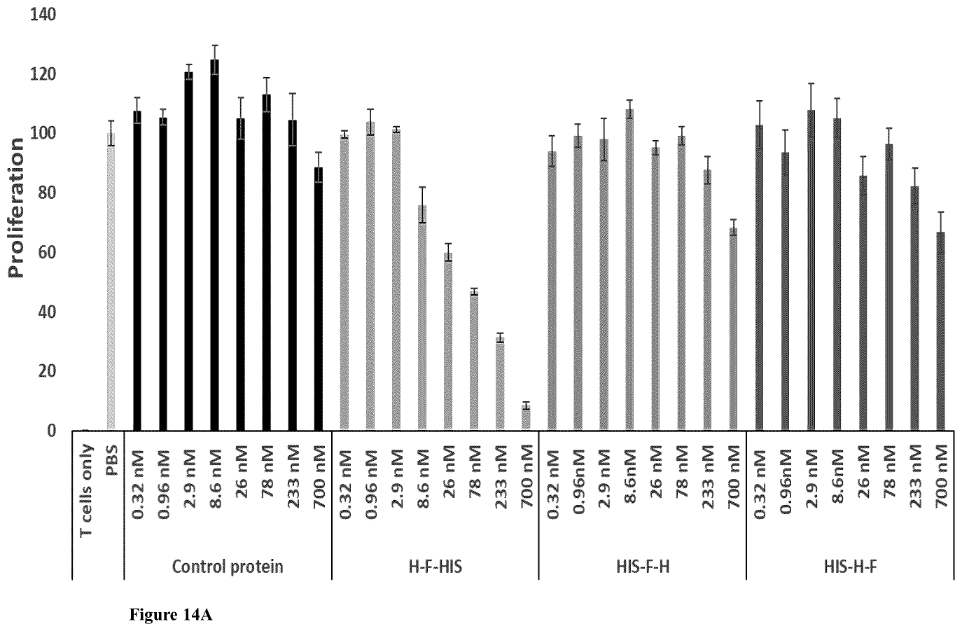

Monocytes were differentiated to mature dendritic cells (DCs) as follows. Monocytes enriched from a healthy human donor were incubated with IL-4 and GM-CSF to induce differentiation toward dendritic cells. On day 7 LPS was added and incubation proceeded for 48 hours in order to generate mature dendritic cells. In order to test the effects of FRED on monocyte differentiation, monocytes at day zero were treated with H-FRED, Fc-FRED (His-monoFc (N297G)-GGGS₂-FRED, 700 nM), control HSA peptide and control Fc peptide which were refreshed throughout the assay. In order to test differentiation, the expression of activation markers CD80 and CD83 were examined by FACS (FIG. 13A). Before differentiation, 94% of immature DCs were not double-positive for CD80 and CD83 but following the protocol 77% of cells were found to be double positive. Control HSA peptide had a minimal and non-significant effect with 57% of cells double positive; similarly control Fc still produced 63% double positive cells. In contrast, H-FRED reduced the percentage of double-positive cells to only 45%; while Fc-FRED was even more effective decreasing the percentage to 25.5% double positive cells.

Secretion of pro-inflammatory cytokines TNFα and IL-6 was also tested in these cells. Both control peptides did not significantly decrease cytokine section, whereas H-FRED, and to an even greater extent Fc-FRED, was effective in inhibiting secretion of both TNFα (FIG. 13B) and IL-6 (FIG. 13C). Thus, H-FRED was capable of inhibiting monocyte differentiation to mature DCs, while Fc-FRED was capable for nearly completely blocking this differentiation.

### Example 16: Reduced Functionality of Other Chimeric Proteins as Compared to H-FRED and FRED-H Proteins

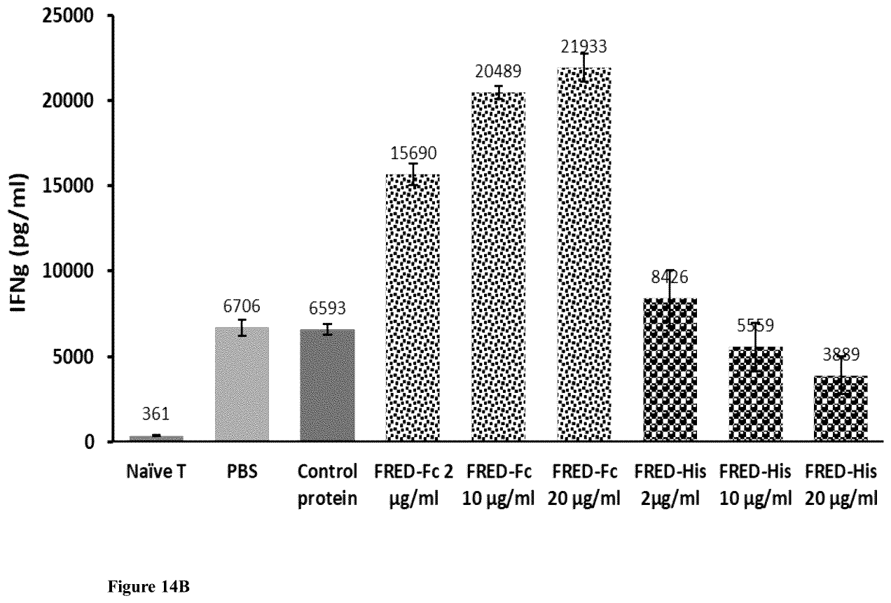

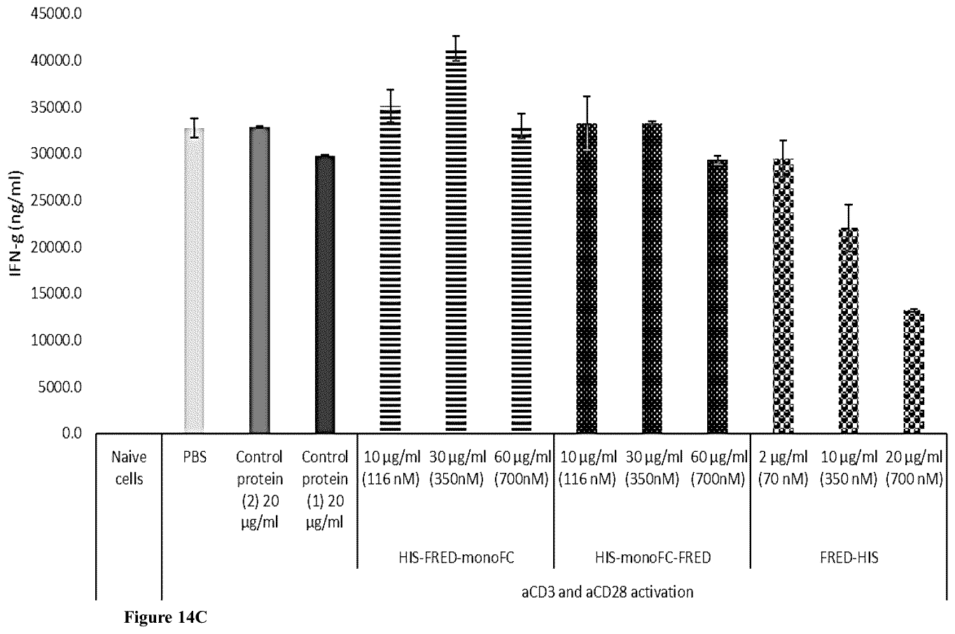

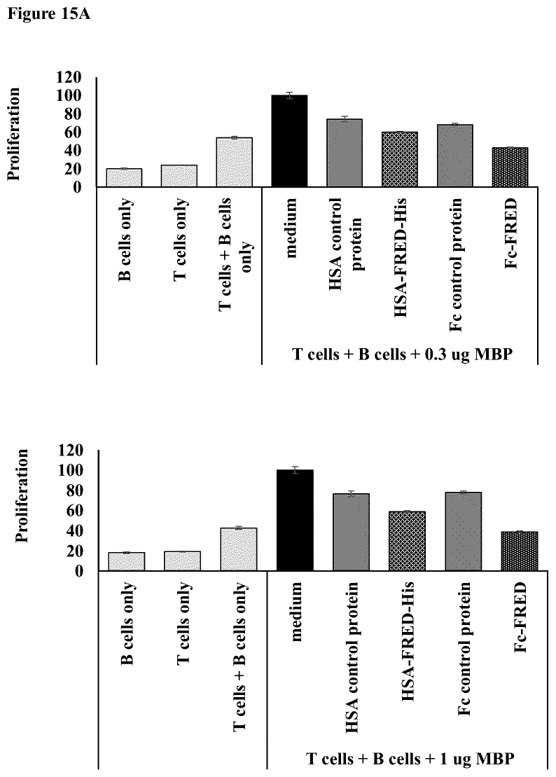

An isolated CD3 positive population from blood samples of healthy donors was activated with anti-CD3 and anti-CD28 antibodies for five days. Recombinant His-FRED-HSA, His-HSA-FRED, H-FRED (H-FRED-His) and FRED-H (FRED-H-His) proteins, were added at 8 concentrations: 700, 233, 78, 26, 8.6, 2.9, 0.96 and 0.32 nM. An irrelevant human protein fused to a His₆ tag, was used as a negative control. Proliferation was determined with an MTT proliferation assay (FIG. 14A). The results show a significantly reduced inhibitory effect with His-FRED-HSA and His-HSA-FRED. Indeed, these molecules, were less effective than FRED-His with no HSA conjugation (FIG. 4A). H-FRED and FRED-H (not shown) were strongly inhibitory, especially at high concentrations (FIG. 14A).

A similar test was performed to measure interferon gamma secretion. An isolated CD3 positive population of cells from blood samples of healthy donors was activated with anti-CD3 and anti-CD28 antibodies for five days. Recombinant FRED-Fc, FRED-His and an irrelevant human protein as negative control, were added at 3 concentrations: 20, 10 and 2 μg/ml (FIG. 14B). The results of FIG. 14B show a significant and clear dose-dependent inhibitory effect of FRED-His while the FRED-Fc did not show any inhibitory effect and even caused T cell activation, as can be seen by increased levels of secreted IFNg. The latter effect may be due the Fc moiety causing dimerization of FRED that may cause immune activation due to cross-linking. Using FRED chimeras with mono-Fc moieties to negate this possible effect exhibited no inhibitory (His-FRED-monoFc) or poor inhibitory (His-monoFc-FRED) effects as compared to H-FRED (FIG. 14C).

### Example 17: H-FRED and MonoFC-FRED Immunomodulatory Function in Multiple Sclerosis Cells

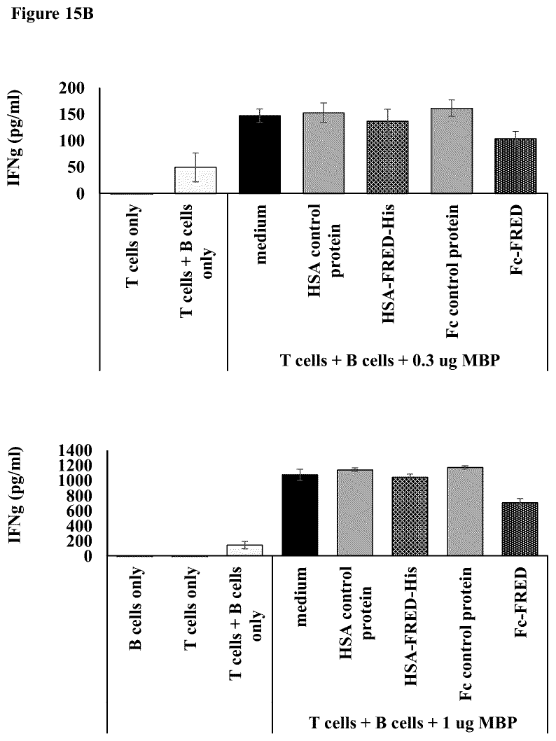

T cell and B cell lines generated from a patient with Multiple Sclerosis (MS) were co-incubated in the presence or absence of two concentrations of the myelin basic protein (MBP, 0.3 or 1 μg) for 5 days. This protein is believed to be the target antigen in MS. The effects of H-FRED and mono-Fc-FRED added at day zero were evaluated. Cell proliferation (FIG. 15A) as measured by MTT assay and INFγ section (FIG. 15B) as measured by ELISA were both decreased, in a dose-dependent manner, with the addition of both molecules. monoFc-FRED was found to be even more potent than H-FRED in this instance.

### Example 18: FRED-H and H-FRED Immunomodulatory Function in Mouse Models of Autoimmune Disease

FRED-H, H-FRED and control protein are administered to a collagen-induced rheumatoid arthritis mouse model and therapeutic effect is measured. The mice's mobility is measured (average arthritis score), as is inflammation (cytokine secretion) and the compounds of the invention are found to increase mobility and decrease inflammation. FRED-H and H-FRED and control protein are administered to a dextran sulfate sodium (DSS) mouse model of colitis and other IBD mouse models. At least one of body weight, rectal bleeding, stool consistency, and survival are measured to assess colitis progression. A disease activity index (DAI) is calculated, and FRED-H and H-FRED improve the DAI score. FRED-H and H-FRED and control protein are administered to NZBXW/F1 female mice having lupus or another lupus mouse model. Cytokine expression is measured to assess disease progression. The compounds of the invention are found to decrease inflammatory cytokine secretion in a lupus model.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An immunomodulatory chimeric molecule, said chimeric molecule comprising:
a fibrinogen-related domain (FRED) from human fibrinogen-like protein 2 (FGL2), or an immunomodulatory fragment or analog thereof, and a half-life extending moiety.

2. The chimeric molecule of claim 1, wherein said half-life extending moiety is selected from human serum albumin (HSA) and monomeric Fc.

3. The chimeric molecule of claim 1, wherein said chimeric molecule is a stronger immunomodulator than said human FRED alone.

4. (canceled)

5. The chimeric molecule of claim 1, to wherein said immunomodulation is immunosuppression and comprises at least one of: reducing secretion of at least one inflammatory cytokine and reducing proliferation of an immune cell.

6. The chimeric molecule of claim 5, wherein said immune cell is selected from a T cell, a B cell and a dendritic cell.

7. The chimeric molecule of claim 1, wherein said half-life extending moiety is conjugated to the N- or C-terminus of said FRED or said immunomodulatory fragment or analog thereof.

8. The chimeric molecule of claim 1, wherein said FRED or said immunomodulatory fragment or analog thereof and said half-life extending moiety are connected by a linker.

9. The chimeric molecule of claim 8, wherein said linker is an amino acid linker.

10. The chimeric molecule of claim 9, wherein said linker comprises the amino acid sequence GGGGS or comprises or consists of the amino acid sequence
(SEQ ID NO: 4)
GGGGSGGGGSGGGGS.

11. (canceled)

12. The chimeric molecule of claim 9, wherein said linker does not comprise a sequence of at least 10 amino acids from FGL2.

13. The chimeric molecule of claim 1, wherein said FRED consists of the amino acid sequence provided in SEQ ID NO: 3.

14. The chimeric molecule of claim 1, further comprising a tag.

15. The chimeric molecule of claim 14, wherein said tag is a His tag, a C-terminal tag or both.

16. (canceled)

17. (canceled)

18. A pharmaceutical composition, comprising a therapeutically effective amount of a chimeric molecule of claim 1 and a pharmaceutically acceptable carrier, excipient or adjuvant.

19. (canceled)

20. A method of reducing inflammation in a subject in need thereof, the method comprising administering to said subject a pharmaceutical composition of claim 18, thereby reducing inflammation in a subject.

21. The method of claim 20, wherein reducing inflammation comprises at least one of: reducing expression of at least one proinflammatory cytokine and reducing proliferation of an immune cell in said subject, said immune cell selected from a T cell and a dendritic cell.

22. The method of claim 21, wherein said proinflammatory cytokine is selected from interferon gamma (IFN-g), tumor necrosis factor alpha (TNFa) and interleukin 6 (IL-6), said reducing expression is reducing expression by an immune cell selected from a T cell and a dendritic cell, or both.

23. (canceled)

24. (canceled)

25. A method of treating an autoimmune disease in a subject in need thereof, the method comprising administering to said subject a pharmaceutical composition of claim 17, thereby treating an autoimmune disease in a subject.

26. The method of claim 25, wherein said autoimmune disease is selected from the group consisting of: rheumatoid arthritis, inflammatory bowel disease, colitis, ulcerative colitis, autoimmune encephalomyelitis (EAE), lupus, Multiple Sclerosis (MS) and Crohn's disease.

27. (canceled)

28. The method of claim 25, wherein treating comprises at least one of:
a. reducing inflammation in said subject;
b. reducing expression of at least one proinflammatory cytokine is said subject;
c. reducing expression of at least one of interferon gamma (IFN-g), tumor necrosis factor alpha (TNFa) and interleukin 6 (IL-6) in said subject;
d. reducing proliferation of an immune cell in said subject, said immune cell being selected from a T cell and a dendritic cell; and
e. reducing differentiation of monocytes to mature dendritic cells in said subject.

29. (canceled)

30. (canceled)

31. (canceled)

32. (canceled)

33. (canceled)


SEQUENCE LISTING (6 sequences)

SEQ ID NO: 1 - protein, 439 aa, Homo sapiens
MKLANWYWLSSAVLATYGFLVVANNETEEIKDERAKDVCPVRLESRGKCEEAGECPYQVSLPPLTIQLPKQFSRIEEVFKEVQNLKEIVNSLKKSCQDCKLQADDNGDPGRNGLLLPSTGAPGEVGDNRVRELESEVNKLSSELKNAKEEINVLHGRLEKLNLVNMNNIENYVDSKVANLTFVVNSLDGKCSKCPSQEQIQSRPVQHLIYKDCSDYYAIGKRSSETYRVTPDPKNSSFEVYCDMETMGGGWTVLQARLDGSTNFTRTWQDYKAGFGNLRREFWLGNDKIHLLTKSKEMILRIDLEDFNGVELYALYDQFYVANEFLKYRLHVGNYNGTAGDALRFNKHYNHDLKFFTTPDKDNDRYPSGNCGLYYSSGWWFDACLSANLNGKYYHQKYRGVRNGIFWGTWPGVSEAHPGGYKSSFKEAKMMIRPKHFKP

SEQ ID NO: 2 - protein, 416 aa, Homo sapiens
NNETEEIKDERAKDVCPVRLESRGKCEEAGECPYQVSLPPLTIQLPKQFSRIEEVFKEVQNLKEIVNSLKKSCQDCKLQADDNGDPGRNGLLLPSTGAPGEVGDNRVRELESEVNKLSSELKNAKEEINVLHGRLEKLNLVNMNNIENYVDSKVANLTFVVNSLDGKCSKCPSQEQIQSRPVQHLIYKDCSDYYAIGKRSSETYRVTPDPKNSSFEVYCDMETMGGGWTVLQARLDGSTNFTRTWQDYKAGFGNLRREFWLGNDKIHLLTKSKEMILRIDLEDFNGVELYALYDQFYVANEFLKYRLHVGNYNGTAGDALRFNKHYNHDLKFFTTPDKDNDRYPSGNCGLYYSSGWWFDACLSANLNGKYYHQKYRGVRNGIFWGTWPGVSEAHPGGYKSSFKEAKMMIRPKHFKP

SEQ ID NO: 3 - protein, 236 aa, Homo sapiens
PVQHLIYKDCSDYYAIGKRSSETYRVTPDPKNSSFEVYCDMETMGGGWTVLQARLDGSTNFTRTWQDYKAGFGNLRREFWLGNDKIHLLTKSKEMILRIDLEDFNGVELYALYDQFYVANEFLKYRLHVGNYNGTAGDALRFNKHYNHDLKFFTTPDKDNDRYPSGNCGLYYSSGWWFDACLSANLNGKYYHQKYRGVRNGIFWGTWPGVSEAHPGGYKSSFKEAKMMIRPKHFKP

SEQ ID NO: 4 - protein, 15 aa, Artificial
  Synthetic
GGGGSGGGGSGGGGS

SEQ ID NO: 5 - protein, 118 aa, Artificial
  Synthetic
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSAAA

SEQ ID NO: 6 - protein, 585 aa, Homo sapiens [sequence omitted]